SEQUENCE LISTING (2 sequences)

SEQ ID NO: 1 - RNA, 426 nt, Mus musculus
  source 1..426: mol_type = mRNA
atgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatga

SEQ ID NO: 2 - DNA, 21 bp, synthetic construct
  misc_feature 1..19: note = RNA
  misc_feature 20..21: note = DNA
  source 1..21: mol_type = other DNA
gctgtacgaggagatattttt

(19) United States
(12) Patent Application Publication
(10) Pub. No.: US 2026/0077063 A1
(43) Pub. Date: Mar. 19, 2026

(54) UNIVERSAL NON-VIRAL GENE DELIVERY SYSTEM WITH ENHANCED STABILITY

(71) Applicants: Brown University, Providence, RI (US); University of Iowa Research Foundation, Iowa City, IA (US)

(72) Inventors: Tejal Desai, Providence, RI (US); Kareem Ebeid, Cranston, RI (US); Brendan Knittle, Pinckney, MI (US); Aliasger Salem, Coralville, IA (US)

(21) Appl. No.: 19/332,428

(22) Filed: Sep. 18, 2025

Related U.S. Application Data
(60) Provisional application No. 63/696,351, filed on Sep. 18, 2024.

(51) Int. Cl. A61K 48/00; A61K 47/59; C12N 15/88

(52) U.S. Cl. CPC A61K 48/0058; A61K 47/593; A61K 47/595; A61K 48/0041; C12N 15/88

(57) ABSTRACT

The present disclosure provides, for instance, polymer-lipid hybrid nanoparticle compositions and methods of making and using them. The polymer-lipid hybrid nanoparticle may comprise, for example, poly(lactic-co-glycolic) acid (PLGA), polyethylenimine (PEI), and D-Lin-MC3-DMA (MC3). The polymer-lipid hybrid nanoparticle can be used to deliver, for example, nucleic acids and small molecules, cells.

### CROSS REFERENCE TO RELATED APPLICATIONS

This application claims the benefit of U.S. Provisional Patent Application No. 63/696,351, filed Sep. 18, 2024. The contents of the aforementioned application are hereby incorporated by reference in their entirety.

### SEQUENCE LISTING

The instant application contains a Sequence Listing which has been submitted electronically in XML format and is hereby incorporated by reference in its entirety. Said XML copy, created on Sep. 16, 2025, is named Tech_ID_3450J_0312021_00256.xml and is 3,798 bytes in size.

### BACKGROUND

Nucleic acids, e.g., mRNA, and small molecules can be utilized in therapeutic methods for treating or preventing disease. Nanoparticles are useful delivery systems for nucleic acids, and small molecules. There is a need in the art for new nanoparticle compositions for use in methods of delivering nucleic acids and small molecules.

### SUMMARY OF THE INVENTION

This disclosure provides, for example, compositions comprising polymer-lipid-hybrid nanoparticles (NPs). These nanoparticles may be used as a delivery vehicle for effectors, such as nucleic acids (e.g., mRNA) and small molecules. This disclosure relates, at least in part, to a polymer-lipid hybrid nanoparticle (NP) platform that employs surface loading of mRNA and can be dried, e.g., lyophilized, into a shelf stable powder. The NPs can be used to encapsulate therapeutic drugs thus enabling the development of dual therapy approaches in which genetic material and drugs can be co-delivered using a single NP delivery system. The compositions described herein may have advantages including stability over time, e.g., a shelf life of at least 2 years, and at higher storage temperatures, e.g., −20° C. The compositions described herein may be used to deliver effectors to cells.

### ENUMERATED EMBODIMENTS

1. A composition comprising:
   (i) poly(lactic-co-glycolic acid) (PLGA);
   (ii) polyethyleneimine (PEI); and
   (iii) D-Lin-MC3-DMA (MC3).
2. The composition of embodiment 1, wherein the PLGA, PEI, and MC3 form nanoparticles.
3. A nanoparticle comprising:
   (i) a non-cationic polymer;
   (ii) a cationic polymer; and
   (iii) D-Lin-MC3-DMA.
4. A nanoparticle comprising:
   (i) poly(lactic-co-glycolic acid) (PLGA);
   (ii) a cationic polymer; and
   (iii) an ionizable lipid.
5. A nanoparticle comprising:
   (i) a non-cationic polymer;
   (ii) poly(amidoamine) (PAMAM) or PEI; and
   (iii) an ionizable lipid.
6. The nanoparticle of embodiment 3 or 5, wherein the non-cationic polymer is PLGA.
7. The nanoparticle of embodiment 3 or 4, wherein the cationic polymer is poly(amidoamine) (PAMAM) or PEI.
8. The nanoparticle of embodiment 4 or 5, wherein the ionizable lipid is D-Lin-MC3-DMA (MC3), SM-102 (SM), or ALC-0315 (ALC).
9. The nanoparticle of any of the preceding embodiments, wherein the mass ratio of PLGA to PAMAM is 50+/−20% to 6.25+/−20%, 25+/−20% to 2.5+/−20%, or 25+/−20% to 1.25+/−20%,
10. The nanoparticle of any of embodiments 1-8, wherein the mass ratio of PLGA to MC3 is 25+/−20% to 5+/−20%, 25+/−20% to 2+/−20%, 25+/−20% to 1.25+/−20%, 25+/−20% to 1+/−20%, 25+/−20% to 0.75+/−20%, 25+/−20% to 0.5+/−20%, 15+/−2⁰% to 1+/−20%, 15+/−20% to 3+/−20%.
11. The nanoparticle of any of embodiments 1-8, wherein the mass ratio of PLGA to PEI to SM is 15+/−20% to 3+/−20% to 1+/−20%.
12. The nanoparticle of any of embodiments 1-8, wherein the mass ratio of PLGA to PEI to ALC is 15+/−20% to 3+/−20% to 1+/−20% or 15+/−20% to 1.5+/−20% to 2+/−20%.
13. The nanoparticle of any of embodiments 1-8, wherein the mass ratio of PLGA to PAMAM to MC3 is 25+/−20% to 2.5+/−20% to 1+/−20%, 25+/−20% to 1.25+/−20% to 1+/−20%, 25+/−20% to 0.05+/−20% to 1+/−20%, or 25+/−20% to 0.5+/−20% to 1+/−20%, or 25+/−20% to 0.25+/−20% to 1+/−20%.
14. The nanoparticle of any of embodiments 1-8, wherein the mass ratio of PLGA to PEI is 25+/−20% to 0.6+/−20%, 25+/−20% to 3+/−20%, 25+/−20% to 6+/−20%, 15+/−20% to 3+/−20%, or 15+/−20% to 1+/−20%,
15. The nanoparticle of any of embodiments 1-8, wherein the mass ratio of PLGA to PEI to MC3 is 15+/−20% to 0.5+/−20% to 1+/−20%, 15+/−20% to 1+/−20% to 1+/−20%, 15+/−20% to 2+/−20% to 1+/−20%, 15+/−20% to 3+/−20% to 1+/−20%, 15+/−20% to 3+/−20% to 3+/−20%, 15+/−20% to 3+/−20% to 1+/−20%, 15+/−20% to 3+/−20% to 2+/−20%, 15+/−20% to 1+/−20% to 2+/−20%, 15+/−20% to 1.5+/−20% to 2+/−20%, 15+/−20% to 1+/−20% to 3+/−20%, 15+/−20% to 0.5+/−20% to 3+/−20%, 15+/−20% to 1.5+/−20% to 2+/−20%.
16. A nanoparticle comprising:
   (i) poly(lactic-co-glycolic acid) (PLGA)
   (ii) polyethyleneimine (PEI); and
   (iii) D-Lin-MC3-DMA (MC3).
17. The composition of embodiment 1 or 2, or the nanoparticle of any of embodiments 3-16, which further comprises a nucleic acid.
18. The composition of embodiment 2, or the nanoparticle of embodiment 17, wherein the nucleic acid is DNA (e.g., pDNA).
19. The composition of embodiment 2, or the nanoparticle of embodiment 17, wherein the nucleic acid is RNA (e.g., mRNA, guide RNA, or siRNA).
20. The composition of embodiment 19, or the nanoparticle of embodiment 19, wherein the mRNA encodes GM-CSF.
21. The composition of embodiment 19, or the nanoparticle of embodiment 19, wherein the mRNA encodes a CRISPR-Cas protein, e.g., a CRISPR-Cas9 protein.
22. The composition of any of embodiments 1, 2, or 17-21, or the nanoparticle of any of embodiments 3-21, which further comprises a small molecule.
23. The nanoparticle of embodiment 22, wherein the small molecule is encapsulated in the nanoparticle.
24. The composition of embodiment 22 or 23, or the nanoparticle of embodiment 22 or 23, wherein the small molecule is a kinase inhibitor, e.g., trametinib.
25. The composition of embodiment 22 or 23, or the nanoparticle of embodiment 22 or 23, wherein the small molecule intercalates with nucleic acid molecules, e.g., doxorubicin.
26. The composition of any of embodiments 1, 2, or 17-25, or the nanoparticle of any of embodiments 3-25, which is in an aqueous solution.
27. The composition of embodiment 2 or 17-26, or the nanoparticle of any of embodiments 3-26, wherein the nucleic acid is located on the surface of the nanoparticle.
28. The composition of any of embodiments 1, 2, or 17-27, or the nanoparticle of any of embodiments 3-27, which is a powder (e.g., a lyophilized powder).
29. The composition of any of embodiments 1, 2, or 17-28, or the nanoparticle of any of embodiments 3-28, which has a mass ratio of about 15+/−20% PLGA:about 1.5+/−20% PEI:about 2+/−20% MC3.
30. The composition of any of embodiments 1, 2, or 17-29, or the nanoparticle of any of embodiments 3-29, wherein the PLGA is poly(D,L-lactide-co-glycolide).
31. The composition of embodiment 30, or the nanoparticle of embodiment 30, wherein the PLGA has a ratio of lactic acid to glycolic acid monomers of 75:25, 50:50, or 85:15.
32. The composition of embodiment 30 or 31, or the nanoparticle of embodiment 30 or 31, wherein the PLGA has a molecular weight of 38-54 kDa.
33. The composition of any of embodiments 1, 2, or 17-32, or the nanoparticle of any of embodiments 3-32, wherein the PEI has an average molecular weight of 25 kDa.
34. The composition of any of embodiments 1, 2, or 17-33, or the nanoparticle of any of embodiments 3-33, wherein the PEI is linear or branched.
35. The composition of any of embodiments 2 or 17-34, or the nanoparticle of any of embodiments 3-34, wherein the nanoparticle has a diameter of 100-200 nm, 120-170 nm, 130-160 nm, 140-150 nm, or 144-148 nm, or about 146 nm.
36. The composition of any of embodiments 2 or 17-35, or the nanoparticle of any of embodiments 3-35, wherein the nanoparticle has a net positive charge.
37. The composition of any of embodiments 2 or 17-36, or the nanoparticle of any of embodiments 3-36, wherein the nanoparticle has a charge (e.g., zeta potential) of 40-70 mV, 45-65 mV, 50-60 mV, or 50-55 mV, or about 53 mV.
38. The composition of any of embodiments 2 or 17-37, or the nanoparticle of any of embodiments 3-37, which has a shelf life of at least 2 years, e.g., at −20° C.
39. The composition of any of embodiments 2 or 17-38, or the nanoparticle of any of embodiments 3-38, which maintains transfection efficiency when stored at −20° C. for at least 6 months, 12 months, 18 months, or 24 months.
40. The composition of any of embodiments 2 or 17-39, or the nanoparticle of any of embodiments 3-39, wherein a nucleic acid complexes rapidly with the nanoparticle.
41. The composition of any of embodiments 2 or 17-40, or the nanoparticle of any of embodiments 3-40, wherein the nanoparticle is double layered.
42. The composition of any of embodiments 2 or 17-41, or the nanoparticle of any of embodiments 3-41, wherein the nanoparticle is capable of delivering a nucleic acid to a cell.
43. The composition of embodiment 42, or the nanoparticle of embodiment 42, wherein the cell is a eukaryotic cell, e.g., a mammalian cell, e.g., a human cell.
44. The composition of any of embodiments 2 or 17-43, or the nanoparticle of any of embodiments 3-43, which does not comprise viral proteins, or fragments thereof.
45. The composition of any of embodiments 2 or 17-44, or the nanoparticle of any of embodiments 3-44, which is immunogenic.
46. The composition of any of embodiments 2 or 17-45, or the nanoparticle of any of embodiments 3-45, which is non-immunogenic or does not induce an immune response.
47. The composition of any of embodiments 2 or 17-46, or the nanoparticle of any of embodiments 3-46, which does not comprise cholesterol.
48. The composition of any of embodiments 2 or 17-46, or the nanoparticle of any of embodiments 3-46, which further comprises cholesterol.
49. The composition of any of embodiments 2 or 17-48, or the nanoparticle of any of embodiments 3-48, wherein the nanoparticle comprises a small molecule and a nucleic acid.
50. The nanoparticle of any of embodiments 3-49, wherein the nucleic acid is protected from nuclease (e.g., RNase or DNase) digestion.
51. The nanoparticle of any of embodiments 3-50, wherein the nucleic acid is complexed with the nanoparticle at a ratio of 1-10, 1-2, 2-4, or 4-10 molecules of mRNA per nanoparticle, or about 8 molecules of mRNA per nanoparticle, about 4 molecules of mRNA per nanoparticle, or about 2 molecules of mRNA per nanoparticle.
52. The nanoparticle of any of embodiments 3-51, wherein the ratio of nanoparticle to nucleic acid by weight is 15+/−20%:1+/−20%, 30+/−20%:1+/−20%, or 60+/−20%:1+/−20%.
53. A kit comprising the composition of any of embodiments 2 or 17-49, or the nanoparticle of any of embodiments 3-52.
54. A kit comprising:
   (i) poly(lactic-co-glycolic acid) (PLGA)
   (ii) polyethyleneimine (PEI); and
   (iii) D-Lin-MC3-DMA (MC3).
55. A container comprising the composition of any of embodiments 2 or 17-49, or the nanoparticle of any of embodiments 3-52.
56. A container comprising the composition of any of embodiments 1, 2, or 18-49, or the nanoparticle of any of embodiments 3-16 or 18-52, and a nucleic acid molecule, wherein the composition or nanoparticle and nucleic acid molecule are a powder.
57. A method of storing the composition of embodiment 28, the nanoparticle of embodiment 28, or the container of embodiment 56, comprising maintaining the composition, nanoparticle, or container at a temperature of −20° C. for 2 years, wherein transfection activity does not drop more than 10%, 20%, 30%, 40%, or 50%.
58. A container or delivery device comprising the composition of embodiment 28, or the nanoparticle of embodiment 28, and an aqueous solution comprising a nucleic acid molecule, wherein the composition or the nanoparticle are separated from the aqueous solution by a breakable septum.
59. The container or delivery device of embodiment 58, wherein the delivery device is a syringe.
60. A method of reconstituting the composition of embodiment 28, or the nanoparticle of embodiment 28, the method comprising adding an aqueous solution to the powder.
61. A method of making the composition of any of embodiments 1, 2, or 17-49, or the nanoparticle of any of embodiments 3-52.
62. A method of delivering a nucleic acid to a cell or tissue, the method comprising contacting the cell or tissue with the composition of any of embodiments 2 or 17-49, or the nanoparticle of any of embodiments 3-52, thereby delivering the nucleic acid.
63. A method of delivering a nucleic acid and a small molecule to a cell or tissue, the method comprising contacting the cell or tissue with the composition of any of embodiments 2 or 17-49, or the nanoparticle of any of embodiments 3-52, thereby delivering the nucleic acid.
64. The method of embodiment 62 or 63, wherein the nucleic acid is DNA (e.g., pDNA).
65. The method of embodiment 62 or 63, wherein the nucleic acid is RNA (e.g., mRNA or siRNA).
66. The method of embodiment 65, wherein the mRNA encodes GM-CSF.
67. The method of embodiment 65 wherein the mRNA encodes a CRISPR-Cas protein, e.g., a CRISPR-Cas9 protein.
68. A method of delivering a small molecule to a cell or tissue, the method comprising contacting the cell or tissue with the composition of any of embodiments 2 or 17-49, or the nanoparticle of any of embodiments 3-52, thereby delivering the nucleic acid.
69. The method of embodiment 68, wherein the small molecule is a kinase inhibitor, e.g., trametinib.
70. The method of embodiment 68, wherein the small molecule intercalates with nucleic acid molecules, e.g., doxorubicin.
71. The method of any of embodiments 68-70, wherein the cell or tissue is in vivo or ex vivo.
72. The method of any of embodiments 68-71, wherein the tissue is heart, lung, liver, spleen, or kidney.
73. The method of any of embodiments 68-71, wherein the cell is a heart cell, a lung cell, a liver cell, a spleen cell, or a kidney cell, or wherein the cell is a cancer cell.
74. A method of treating a disease or disorder, comprising administering the nanoparticle of any of embodiments 3-52 to a subject.
75. The method of embodiment 74, wherein the disease or disorder is cancer, e.g., melanoma.
76. The method of embodiment 74 or 75, wherein the nanoparticle is administered systemically, e.g., (intravenously, intramuscularly, or subcutaneously) or locally (e.g., intratumorally).

### BRIEF DESCRIPTION OF THE DRAWINGS

FIG. 1 is a schematic depicting storage and reconstitution of NPs: (top) Powdered NPs are added alongside powdered genetic material in the same vial. Upon reconstitution, and simple handshaking the delivery system complexes the genetic material and becomes ready for administration. (bottom) powdered NPs and liquid genetic material are packaged separately in a dual compartment syringe and mixed just before delivery.

FIG. 2 is a schematic depicting the nanoparticle production process. The organic polymer-lipid and drug mixture is added to a syringe and passively dripped into the aqueous phase of 0.5% polyvinyl alcohol (surfactant solution). After the surfactant-stabilized nanoparticle formulation is made, the organic solvent is evaporated, e.g., using a rotary evaporator. The solution is then subjected to centrifugal ultra-filtration twice. After the first two centrifugations, the top chamber of the centrifugal concentrator is topped up to 10 mL to wash the NPs and remove unreacted synthesis materials. The nanoparticle solution is then lyophilized in the presence of a cryoprotectant, e.g., sucrose, to form a nanoparticle powder.

FIG. 3 shows an exemplary flow cytometry gating strategy for detecting GFP expressing HEK293T (top) and B16F10 (bottom) cells. First, live cells were selected based on forward scatter (FSC) and side scatter (SSC) (left panel). Single cells were selected based on FSC height (FSC-H) and area (FSC-A) (left middle panel). For HEK293T untreated cells, 0.069% of the gated cells showed GFP expression (right middle panel), while in cells transfected with GFP using lipofectamine, 97.1% of the gated cells show GFP expression (right panel). For B16F10 untreated cells, 0.51% of the gated cells showed GFP expression (right middle panel), while in cells transfected with GFP using lipofectamine, 93.6% of the gated cells show GFP expression (right panel)

FIGS. 4A-4F are bar plots showing transfection efficiency of exemplary formulations. In FIG. 4A, PLGA+PAMAM and PLGA+PEI formulations were tested with or without MC3 (lipid). An increase in transfection was achieved when PEI was used in the presence of the ionizable lipid. In FIG. 4B, PLGA+PEI formulations were tested with SM, ALC, or MC3 lipids. MC3 had the highest transfection efficiency. In FIG. 4C, the PLGA+PEI+MC3 nanoparticles were made with PEI added either during the aqueous or organic phase. PEI addition in the organic phase resulted in higher transfection efficiency. In FIG. 4D, different amounts of MC3 were tested. Higher amounts of MC3 resulted in higher transfection efficiency, as well as an IC50 of about 80 ug/mL (rightmost bar) or 50 ug/mL (middle bar), compared to an IC50 of about 100 ug/mL (leftmost bar). In FIG. 4E, the amount of PEI was changed. Higher amounts of PEI resulted in higher transfection efficiency, as well as an IC50 of about 50 ug/mL (rightmost bar) about. In FIG. 4F, ratios of PEI:lipid at 3:1, 1.5:2, and 1:3 were tested. The ratio of 1.5:2 had the highest transfection efficiency with high cell viability, as indicated by an IC50 of about 250 ug/mL. From left to right bars correspond to NP batches 4, 5, 36, 55, 53, 54, 55, 55, 56, 56, 59, 60, 60, 66, 67, 56, 63, and 66 according to Table E1.

FIG. 5A is a bar plot showing dynamic light scattering (DLS) measurements of particle size (diameter in nm, circles) and zeta potential (mV, squares) of NPs before and after lyophilization and when complexed with mRNA at Ratios A, B, and C. These measurements were similar between different amounts of mRNA and before and after lyophilization. NP71 denotes nanoparticle 71 according to Table E1. FIG. 5B shows gel electrophoresis images of NPs before and after lyophilization and when complexed with mRNA at Ratios A, B, and C. No mRNA traveled down the gel in the nanoparticle+mRNA wells, showing that the nanoparticles are complexed with the mRNA. FIG. 5C shows TEM images of fresh NPs with size distribution insert (above) and SEM image of fresh NPs with a higher magnification insert (below). FIG. 5D is a bar plot showing DLS measurements of particle size (diameter in nm, circles) and zeta potential (mV, squares) of NPs with and without encapsulated drugs (trametinib or doxorubicin).

FIG. 6 depicts exemplary distributions of hydrodynamic size and zeta potential of NPs with and without encapsulated drugs (trametinib and doxorubicin). Using a stock trametinib concentration of 25 mg/mL, no significant difference in size or zeta potential were observed. Doxorubicin was used at a stock concentration of 500 mg/mL.

FIG. 7A is a bar plot showing dynamic light scattering (DLS) measurements of particle size (diameter in nm, circles) and zeta potential (mV, squares) of NPs after lyophilization without sucrose. FIG. 7B is a bar plot of transfection efficiency. NPs lyophilized in the presence of sucrose had a slightly higher transfection efficiency. FIG. 7C shows gel electrophoresis images of NPs after lyophilization without sucrose. No mRNA traveled down the gel in the nanoparticle+mRNA wells, showing that the nanoparticles are complexed with the mRNA after lyophilization without sucrose. FIG. 7D shows TEM images of NPs lyophilized with and without sucrose. The NPs lyophilized without sucrose were larger than those lyophilized with sucrose.

FIG. 8A is a bar plot showing the transfection efficiency of NP71 and lipid nanoparticles (LNPs). NP71 exhibited equivalent transfection efficiency to that of LNPs in HEK293 cells. FIG. 8B shows representative fluorescent microscopy images of HEK293T cells transfected with GFP-mRNA using NP71 or LNPs following a 48 hour incubation. Scale bars=200 μm.

FIG. 9A is a gel electrophoresis image showing exemplary mixing times of 10 second, 3 seconds, or no mixing, and methods (vortexed or mixed by hand). All mixing times and methods resulted in mRNA complexation. FIG. 9B is a gel electrophoresis image showing protection of mRNA complexed to NPs from an exemplary nuclease. The first lane shows mRNA alone; the second lane shows mRNA+nuclease; the third lane shows mRNA-NPs; the fourth lane shows mRNA-NPs+heparin; the fifth lane shows mRNA-NPs+nuclease; the sixth lane shows mRNA-NPs+nuclease+heparin. In the mRNA-NPs+nuclease lane, mRNA is detected in and near the well, showing that mRNA is not degraded by the nuclease when complexed with the nanoparticle. All mRNA was complexed to NPs at Ratio A using 1 μg of mRNA.

FIG. 10A is a gel electrophoresis image showing that binding activity of NPs to mRNA is unaffected by doxorubicin loading status. Doxorubicin NPs are capable of binding mRNA at ratios A and B, similar to empty NPs. FIG. 10B is a gel electrophoresis image showing that binding activity of NPs to mRNA is unaffected by trametinib loading status. Trametinib NPs both with and without lipids are capable of binding mRNA at ratios A and B, similar to empty NPs.

FIG. 11 shows representative SEM Images of Dox-NPs (left) and Tram-NPs (right). The drug-loaded NPs were morphologically similar in size, shape, and uniformity to unloaded NPs. Dox-NPs appear to be slightly smaller in size, which is expected based on the hydrodynamic size of these NPs when measured through DLS.

FIG. 12 shows exemplary calibration curves of trametinib (left) and doxorubicin (right) concentration versus HPLC UV-Vis peak area, with r²=0.9999. Trametinib absorbance was recorded at 250 nm and doxorubicin absorbance was recorded at 230 nm. Calibration curves were generated each time HPLC was used for measuring drug encapsulation.

FIG. 13 shows dose-response curves oftrametinib (left) and doxorubicin (right) to B16F10. Viability of cells was normalized to the average of an untreated control group. Coefficients of determination correspond to a goodness of fit for a three-parameter nonlinear regression using GraphPad Prism.

FIG. 14 shows an exemplary flow cytometry gating strategy for detecting Cy5 expressing HEK293T cells. First, live cells were selected based on forward scatter (FSC) and side scatter (SSC) (left panel). Single cells were selected based on FSC height (FSC-H) and area (FSC-A) (left middle panel). For untreated cells, 1.04% of the gated cells showed Cy5 expression (right middle panel), while in cells exposed to Cy5 mRNA-NPs for four hours, 99.7% of the gated cells show Cy5 expression (right panel).

FIG. 15 is a plot showing dose-response curves of B16F10 exposed to DMSO using equivalent amounts contained in the doses of drugs given in viability experiments. The x-axis represents the log of the drug concentration, with each point corresponding to the viability of cells exposed to the equivalent amount of DMSO used at that concentration. The presence of DMSO had no significant impact on the viability of these cells.

FIG. 16A shows NP dose-response curves for B16F10 cell viability versus ratio of cells to weight mRNA in ug. FIG. 16B shows NP dose-response curves for B16F10 cell viability versus mRNA concentration. Dotted vertical lines indicate the equivalent number of cells per g of mRNA that corresponds to the indicated dose of mRNA for 24-well plates.

FIG. 17 NP dose-response curves for B16F10 cell viability versus the ratio of number of cells to the molar concentration of trametinib after 48 hours of incubation, the IC50 of the Tram-NPs was 1.39 M.

FIG. 18A shows exemplary histograms of nanoparticle uptake in HEK293T measured through flow cytometry. Histograms from left to right correspond to increasing NP incubation times on cells as shown by the legend insert. Below the histogram groups are bar charts of the median fluorescence intensities. FIG. 18B shows exemplary histograms of nanoparticle uptake in HEK293T measured through flow cytometry. Dot shading indicates curves correlating to a dose of 0.5 μg, curved line shading indicates curves correlating to a dose of 1 μg, and dot and diagonal line shading indicates curves correlating to a dose of 2 μg. Below the histogram groups are bar charts of the median fluorescence intensities.

FIG. 19 shows confocal fluorescence microscopy images demonstrating that mRNA-NPs can escape the endosomes ofHEK293T (top) and B16F10 (bottom) cells. Images show Cy5 (left), nucleus (left middle), lysosome (right middle), and combined (right) signals. Scale bars are 20 μm. There was significant presence of Cy5 signal outside of the lysosome signal, indicating that mRNA-NPs taken up by cells were able to escape the endosome and reach the cytoplasm.

FIG. 20 shows confocal fluorescence microscopy images demonstrating the ability for trametinib-loaded mRNA-NPs to escape the endosomes of B16F10. B16F10 untreated cells are shown on the top row; B16F10 cells treated with tram-NPs with Cy5 mRNA are shown on the bottom row. Images show Cy5 (left), nucleus (left middle), lysosome (right middle), and combined (right) signals. The presence of Cy5 signal outside of the lysosome signal in the cells treated with Tram-NPs indicates that trametinib-loaded mRNA-NPs taken up by cells are able to escape endosomes and reach the cytoplasm. Scale bars are 20 μm.

FIG. 21A shows exemplary brightfield (left) and GFP (right) images of HEK293T cells at 48 hours following transfection with GFP mRNA-NPs. Images were taken on a fluorescence microscope. FIG. 21B is a bar graph showing transfection efficiency of mRNA only (blank) NPs and Tram-NPs (B3:unloaded NPs at Ratio B, total dose 3 μg mRNA; B2: Tram-NPs at Ratio B, total dose 2 μg). Significance testing was performed using student's t-test. FIG. 21C is a bar graph of the transfection efficiency of GM-CSF mRNA NPs (unloaded) and GM-CSF mRNA-Tram-NPs. GM-CSF mRNA-Tram-NPs were able to transfect B16F10 cells with GM-CSF mRNA. These data show that mRNA-NPs can transfect HEK293T and B16F10 cells with GFP and GM-CSF mRNA.

FIG. 22 is a bar plot showing ELISA results of GM-CSF transfection of B16F10 cells using empty and doxorubicin-loaded mRNA NPs. B(1) and B(2) correspond to blank (or empty) NPs and doxorubicin-loaded NPs at Ratio B giving a dose of 1 or 2 μg of GM-CSF mRNA. NPs loaded with doxorubicin were able to transfect cells with GM-CSF. Cells were seeded at 5000 cells/well for this experiment.

FIG. 23A shows the treatment scheme for the in vivo mouse study. Mice are challenged with B16F10 cells by injection at day 0. Treatments are injected intratumorally at days 9, 11, 13, 15, and 17. FIG. 23B shows representative photographs of individual mice in each group (untreated, blank NP, Tram NP, Tam NP+GM-CSF mRNA) with their tumor areas circled. FIG. 23C is a plot showing tumor volume (in mm³) and time post tumor challenge (in days) in each group (untreated, blank NP, Tram NP, Tam NP+GM-CSF mRNA). Tram NPs+GM-CSF mRNA inhibited tumor growth over time. Curves were stopped when any individual in the group reached the tumor growth endpoint. FIG. 23D is a plot showing overall survival % and time post-tumor challenge (days) in each group (untreated, blank NP, Tram NP, Tam NP+GM-CSF mRNA). Tram NPs+GM-CSF mRNA improved survival in mice. Significance was determined using the logrank test between all treatment groups and the untreated control group. FIG. 23E is a plot showing weight in grams and time post tumor challenge in days for each group (untreated, blank NP, Tram NP, Tam NP+GM-CSF mRNA). Mice weights remained unchanged throughout the duration of the study. These data show that NPs significantly inhibit melanoma growth and improve survival in vivo.

FIG. 24 is a bar plot showing median survival in days of mice in each treatment group (untreated, blank NP, Tram NP, Tam NP+GM-CSF mRNA). The combination therapy decreased tumor growth over time and increased survival compared to both untreated mice and mice that received Tram-NPs without mRNA. Significance in was determined using the log-rank test.

FIG. 25 (top) is a bar plot showing the transfection efficiency of NPs loaded with 1 ug or 2 ug of plasmid DNA encoding eGFP, at a ratio of 15 ug NPs to 1 ug mRNA. The transfection efficiency is similar for NPs loaded with 1 ug or 2 ug. (bottom) is a flow cytometry plot showing that untreated cells were not positive for GFP expression, while cells treated with NPs loaded with 1 ug or 2 ug of plasmid DNA encoding eGFP were positive for GFP expression.

FIG. 26 (top) is a bar graph of luminescence in HEK293T untreated cells, cells treated with luciferase mRNA-NPs (SUN_{DP}) with 1 ug, 2 ug, or 3 ug of mRNA, or cells treated with 1 ug luciferase mRNA only. Cells treated with the luciferase mRNA-NPs exhibited luminescence, indicating successful luciferase transfection. (bottom) an image showing a luciferase expression assay on untreated cells, cells treated with luciferase mRNA-NPs with 1 ug, 2 ug, or 3 ug of mRNA, or cells treated with 1 ug luciferase mRNA only. Untreated cells and mRNA only cells did not show luciferase expression. Cells treated with luciferase mRNA-NPs expressed luciferase. These results show that NPs are able to deliver luciferase mRNA, which is a longer mRNA of 1921 nucleotides.

FIG. 27 shows the different ratios of NPs to mRNA. Ratio A: 15 ug NPs complexed with 1 ug mRNA results in 8 mRNA molecules per NP, on average. Ratio B: 30 ug NPs complexed with 1 ug mRNA results in 4 mRNA molecules per NP, on average. Ratio C: 60 ug NPs complexed with 1 ug mRNA results in 2 mRNA molecules per NP, on average.

FIG. 28 is a bar graph showing transfection efficiencies of lyophilized batches of NP #58A (Table 2) complexed with GFP-mRNA and evaluated against HEK 293T cells at different complexation ratios (ratio A, B, and C as shown in FIG. 29) and mRNA doses following a 48 h incubation via flow cytometry. Transfection efficiencies were similar between NPs lyophilized with sucrose and NPs lyophilized without additives. Statistical analysis was carried out using one-way ANOVA with a Tukey post hoc test. Data are expressed as mean±s.d. (n=3). ***P<0.001.

FIG. 29 is a bar graph showing the relative performance of NPs and LNPs after storage in various conditions. Lyophilized NPs (SUN_{DP}) were stored at −20 C, 4 C, and 20 C for six months. Lipid nanoparticles (LNPs) were stored at 4 C for 1 month. Stored NPs and LNPs were used to transfect cells with GFP mRNA. The stored NPs maintained transfection efficiency compared to fresh NPs, while stored LNPs had reduced transfection efficiency compared to fresh LNPs. Statistical analysis was carried out using repeated-measures one-way ANOVA with the Gessier-Greenhouse correction with a Tukey post hoc test for comparisons between SUNDP groups and paired two-sided t-test for comparisons between LNP groups.

FIG. 30 is a plot showing cell viability and NP concentration (ug/ml). Cytotoxicity of NPs (SUN_{DP}) alone or complexed with GFP-mRNA at Ratio A were incubated with HEK 293T cells for 48 h. Vertical lines represent the equivalent NP doses used at the indicated ratios (A, B, and C) for transfection studies. Cell viability was assessed using the MTS assay. Cell viability data was fitted to curves using nonlinear regression of dose-response inhibition with variable slope utilizing GraphPad Prism. Statistical analysis between the IC50 values was carried out using paired two-sided t-test. NPs with and without mRNA had similar effects on cell viability.

FIG. 31 (top) is plot of histograms showing the Cy5 fluorescence of HEK293T cells treated with Cy5 mRNA-NPs (SUN_{DP}), where NPs were complexed to 1 ug of Cy5-Luc-mRNA at Ratio A. Cells were either untreated or exposed to NPs for 5, 10, 30, 60, 120, or 240 minutes. (bottom) is a bar graph showing median fluorescence intensity of cells which were untreated or exposed to NPs for 5, 10, 30, 60, 120, or 240 minutes. Median fluorescence of cells increased as the time of exposure increased. Uptake was measured using flow cytometry.

FIG. 32 (top) is a plot of histograms showing the Cy5 fluorescence of HEK293T cells either untreated or treated with Cy5 mRNA-NPs (SUN_{DP}) complexed with mRNA at ratio A (left), B (middle), or C (right), at doses equivalent to 0.5 ug total mRNA, 1 ug total mRNA, or 2 ug total mRNA, after 1 hour of incubation. (bottom) a bar plot showing median fluorescence intensity of the histograms shown in the top panel. The median fluorescence intensity increased with amount of mRNA in the treatment.

FIG. 33 is a confocal microscopy image demonstrating the ability of NPs (SUN_{DP}) to deliver mRNA outside of the lysosomal compartments of HEK293T. Cy5 mRNA (top left panel), nucleus (top middle panel), lysosome (top right panel) and merge (bottom panel) are shown. The top white arrow indicates an example of a lysosome with entrapped NPs and the bottom arrow indicates an example of a lysosome without entrapped NPs. Scale bars are 10 um.

FIG. 34 (top) is a bar plot showing the transfection efficiency of GFP mRNA-NPs at ratio A (SUN_{DP} (A)) with 1, 2, or 3 ug total mRNA, or ratio B (SUN_{DP} (B)), with 1.5 or 2 ug total mRNA, in APRE-19 cells. (bottom) a plot showing GFP positive and negative cells after transfection as measured by flow cytometry. NPs were able to transfect APRE-19 cells, a human cell line.

FIG. 35 (top) is a bar plot showing the transfection efficiency of GFP mRNA-NPs at ratio A (SUN_{DP} (A)) with 1 or 2 ug total mRNA, or ratio B (SUN_{DP} (B)), with 1, 1.5, or 2 ug total mRNA, in B16F10 cells. (bottom) a plot showing GFP positive and negative cells after transfection as measured by flow cytometry. NPs were able to transfect B16F10 cells, a mouse cell line.

FIG. 36 (top) is a western blot of SIRT1 in cells which where untreated, treated with SIRT1 siRNA alone, treated with NP (SUN_{DP}) alone, treated with NP and siRNA (SUN_{DP}+siRNA), or treated with LF (lipofection)+siRNA. Treatment with NP and siRNA reduced expression of SIRT1 in cells. (bottom) a bar graph showing percent SIRT1 expression in the conditions shown in the top panel. NP+siRNA reduced SIRT1 protein as much as lipofection of the siRNA. **P<0.01, ns, not significant.

FIG. 37 (top) a schematic of the biodistribution study. Mice were retroorbitally injected with NPs loaded with DIR dye (DIR-SUN_{DP}) or (PBS). The brain, heart, lung, liver, spleen, kidney, and muscle were collected 24 hours after injection. (bottom) The image on the left is DIR-SUNDP in a tube by itself. Imaging of the organs showed that DIR-SUN_{DP} was present in the heart, lung, liver, spleen, and kidney.

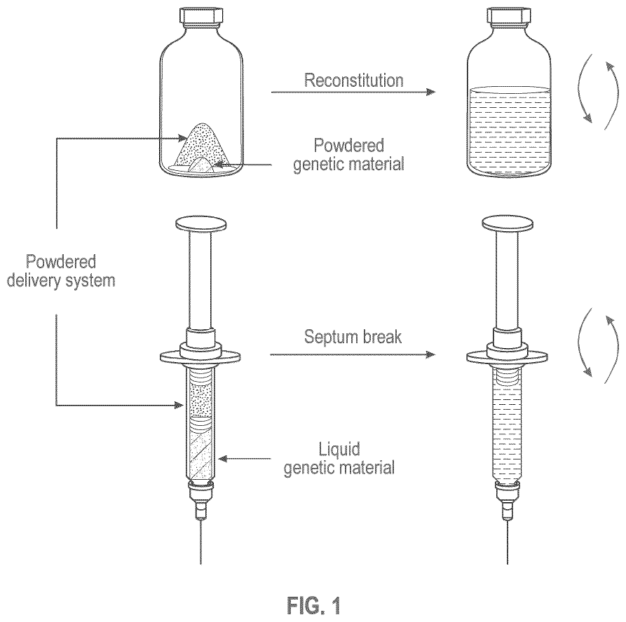

### DETAILED DESCRIPTION OF THE INVENTION

The present disclosure provides, for example, polymer-lipid-hybrid nanoparticle compositions and methods of using same.

In the following description, for an explanation, numerous specific details provide a thorough understanding of the compositions and methods disclosed herein. However, it may be evident that the compositions and methods may be practiced without these specific details. Aspects, modes, embodiments, variations, and features of the compositions and methods are described below in various levels of detail to provide a substantial understanding of the present disclosure.

### Definitions

For convenience, the meaning of some terms and phrases used in the specification, examples, and appended claims, are listed below. Unless otherwise defined, all technical and scientific terms have the same meaning as commonly understood by a person having ordinary skill in the biomedical art to which this invention belongs. A term's meaning provided in this specification shall prevail if any apparent discrepancy arises between the meaning of a definition provided in this specification and the term's use in the biomedical art.

The singular forms a, an, and the like include plural referents unless the context dictates otherwise. For example, a reference to a cell comprises a combination of two or more cells.

As used herein, the term “comprising” means that other elements can also be present in addition to the defined elements presented. Using comprising indicates inclusion rather than limitation.

As used herein, the term “consisting essentially of” means the listed elements are required for a given embodiment. The term permits additional elements that do not materially affect the basic and functional characteristics of that embodiment of the invention.

As used herein, the term “consisting of” means compositions, methods, and respective components thereof, exclusive of any element not recited in that description of the embodiment.

As used herein, the term “nucleic acid” refers to a polymeric molecule incorporating units of ribonucleic acid, deoxyribonucleic acid, or an analog thereof. In some embodiments, the nucleic acid is in single stranded form. In some embodiments, the nucleic acid is in double stranded form. In some embodiments, the nucleic acid is genomic DNA, cDNA, or RNA (e.g. mRNA). In some embodiments, the nucleic acid contains analogues of natural nucleotides that have similar binding properties as the reference nucleic acid. In some embodiments, the nucleic acid containing analogues of natural nucleotides are metabolized in a manner similar to naturally occurring nucleotides.

As used herein, the term “or” refers to and/or. The term and/or as used in a phrase such as A and/or B herein includes both A and B; A or B; A (alone); and B (alone). Likewise, the term and/or as used in a phrase such as A, B, and/or C encompasses each embodiment: A, B, and C; A, B, or C; A or C; A or B; B or C; A and C; A and B; Band C; A (alone); B (alone); and C (alone).

As used herein, the terms “peptide,” “polypeptide,” and “protein” are used interchangeably, and refer to a molecule comprised of two or more amino acid residues covalently linked by peptide bonds between the alpha-amino and carboxy groups of adjacent residues. In some embodiments, the polypeptide comprises a modified amino acid. In some embodiments, the polypeptide refers to a natural peptide, a recombinant peptide, or a combination thereof. In some embodiments, the polypeptide refers to short chains of amino acids. In some embodiments, the polypeptide refers to long chains of amino acids. In some embodiments, the polypeptide refers to a biologically active fragment, a substantially homologous polypeptide, an oligopeptide, a variant of a polypeptide, a modified polypeptide, a derivative, an analog, or a fusion protein. A person having ordinary skill in the biomedical art recognizes that individual substitutions, deletions, or additions to a peptide, polypeptide, or protein sequence which alters a single amino acid or a small percentage of amino acids in the encoded sequence are a conservatively modified variant where the alteration results in the substitution of amino acid with chemically similar amino acid and retains the desired activity of the polypeptide. Such conservatively modified variants also do not exclude polymorphic variants, interspecies homologs, and alleles consistent with the disclosure.

As used herein, the term “subject” refers to a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or geriatric subject. The human subject may be of either sex. In some embodiments, the subject may have a condition or disease or be at risk of developing a condition or disease.

This invention is not limited to the particular methodology, protocols, reagents, etc., described herein and as such can vary.

The disclosure described herein does not concern a process for cloning humans, processes for modifying the germ line genetic identity of humans, uses of human embryos for industrial or commercial purposes, or processes for modifying the genetic identity of animals likely to cause them suffering with no substantial medical benefit to man or animal, and animals resulting from such processes.

### Nanoparticle Compositions

In some embodiments, the NPs comprise a non-cationic polymer, a cationic polymer, and an ionizable lipid. In some embodiments, the NPs comprise a formulation of Table 1, Table 2, Table 3, or Table E1. In some embodiments, the NPs are formulated according to line 66A of Table 2.

### Non-Cationic Polymers

In some embodiments, the non-cationic polymer is poly(lactic-co-glycolic) acid (PLGA). In some embodiments, PLGA comprises the structure according to formula I:

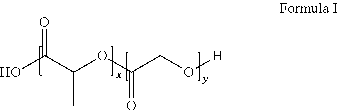

wherein x is the number of units of lactic acid and y is the number of units of glycolic acid. In some embodiments, the PLGA further comprises PEG (PLGA-PEG). In some embodiments, the PLGA has a ratio of about 90+/−20%:10+/−20%, 80+/−20%:20+/−20%, 70+/−20%:30+/−20%, 60+/−20%:40+/−20%, 50+/−20%:50+/−20%, 40+/−20%:60+/−20%, 30+/−20%:70+/−20%, 20+/−20%:80+/−20%, or 10: +/−20%:90+/−20% lactic acid to glycolic acid monomers. In some embodiments, the PLGA comprises 0-10%, 10-20%, 20-30%, 30-40%, 40-50%, 50-60%, 60-70%, 70-80%, 80-90%, or 90-100% lactic acid monomers by weight. In some embodiments, the PLGA comprises 0-10%, 10-20%, 20-30%, 30-40%, 40-50%, 50-60%, 60-70%, 70-80%, 80-90%, or 90-100% glycolic acid monomers by weight. In some embodiments, the non-cationic polymer is polylactic acid (PLA), e.g., does not comprise glycolic acid. In some embodiment, the non-cationic polymer is polyglycolide (PGA), e.g., does not comprise lactic acid.

### Cationic Polymers

In some embodiments, the cationic polymer is polyethylenimine (PEI). In some embodiments, PEI comprises the structure according to Formula II:

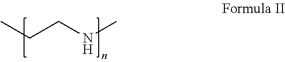

In some embodiments, the PEI is linear. In some embodiments, the PEI is branched. In some embodiments, the PEI comprises primary, secondary and/or tertiary amino groups. In some embodiments, the PEI has an average molecular weight of about 0.5-2000 kDa, 0.5-1 kDa, 1-10 kDa, 10-15 kDa, 15-20 kDa, 20-25 kDa, 25-30 kDa, 30-35 kDa, 35-50 kDa, 50-100 kDa, 100-200 kDa, 200-300 kDa, 300-400 kDa, 400-500 kDa, 500-600 kDa, 600-700 kDa, 700-800 kDa, 800-900 kDa, 900-1000 kDa, 1000-1100 kDa, 1100-1200 kDa, 1200-1300 kDa, 1300-1400 kDa, 1400-1500 kDa, 1500-1600 kDa, 1600-1700 kDa, 1700-1800 kDa, 1800-1900 kDa, or 1900-2000 kDa. In some embodiments, the PEI has an average molecular weight of 0.5, 1, 10, 15, 20, 25, 30, 35, 50, 100, 200, 300, 400, 500, 600, 700, 800, 900, 1000, 1100, 1200, 1300, 1400, 1500, 1600, 1700, 1800, 1900, or 2000 kDa.

In some embodiments, the cationic polymer is poly(amidoamine) (PAMAM). In some embodiments, the PAMAM is a dendrimer. In some embodiments, the PAMAM comprises repetitively branched subunits of amide and amine functionality. In some embodiments, the PAMAM comprises anethylenediamine core. In some embodiments, the PAMAM is 5^{th} generation. In some embodiments, the PAMAM has the linear formula [NH2(CH2)2NH2]:(G=5); dendri PAMAM(NH2)128.

### Ionizable Lipids

In some embodiments, the ionizable lipid is D-Lin-MC3-DMA (MC3) (e.g., (6Z,9Z,28Z,31Z)-Heptatriaconta-6,9,28,31-tetraen-19-yl 4-(dimethylamino)butanoate). In some embodiments, D-Lin-MC3-DMA comprises the structure according to Formula III:

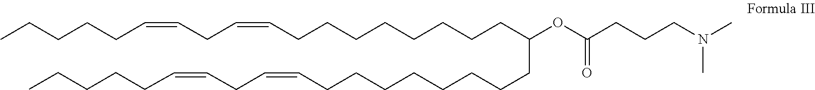 [SMILES: CCCCC/C=C\C/C=C\CCCCCCCCC(CCCCCCCC/C=C\C/C=C\CCCCC)OC(=O)CCCN(C)C]

In some embodiments, the ionizable lipid is SM-102 (SM). In some embodiments, SM-1021 comprises the structure according to Formula IV:

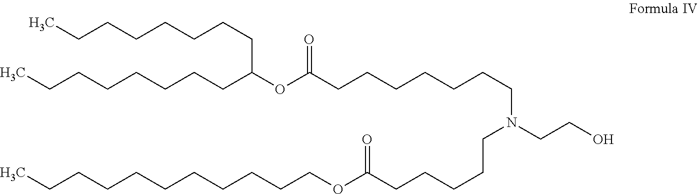 [SMILES: CCCCCCCCCCCOC(=O)CCCCCN(CCO)CCCCCCCC(=O)OC(CCCCCCCC)CCCCCCCC]

In some embodiments, the ionizable lipid is ALC-0315 (ALC) (e.g., [(4-Hydroxybutyl)azanediyl]di(hexane-6,1-diyl) bis(2-hexyldecanoate)). In some embodiments, ALC-0315 comprises the structure according to Formula V:

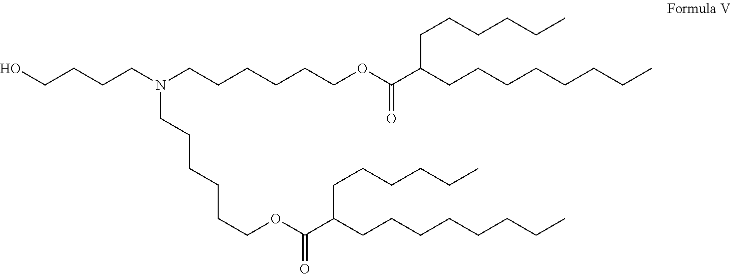 [SMILES: CCCCCCCCC(CCCCCC)C(=O)OCCCCCCN(CCCCO)CCCCCCOC(=O)C(CCCCCC)CCCCCCCC]

In some embodiments, the NPs have a cationic charge. In some embodiments, the cationic charge enhances stability in solution. In some embodiments, the cationic charge increases the interaction with the cell membrane, e.g., the polar headgroups of the cell membrane lipids. In some embodiments, the NPs are able to escape the endosome.

### Effectors

In some embodiments, an effector described herein comprises a nucleic acid or a small molecule. The nanoparticles described herein may be used to deliver the effector to a target cell or tissue.

### Nucleic Acids

In some embodiments, nucleic acids are adsorbed onto the surface of the NPs (e.g., forming a polyplex). In some embodiments, the nucleic acid and the NPs are connected through electrostatic complexation. In some embodiments, the nucleic acid is negatively charged, e.g., due to the phosphate groups in the sugar phosphate backbone. In some embodiments, the surface of the NP is positively charged, e.g., due to comprising a cationic polymer (e.g., PEI or PAMAM). Without wishing to be bound by theory, as the RNA is usually physically entangled with the cationic surface components of the NP following adsorption, nucleases and other degradative enzymes typically cannot bind to the attached RNA to initiate degradation. In some embodiments, the polyplex releases RNA transcripts once inside a cell. In some embodiments, polyplexes are formed through mixing of polymers and RNA in solution. In some embodiments, a surfactant is used to stabilize assembly of polyplexes.

In some embodiments, the nucleic acid is RNA, e.g., mRNA or siRNA. In some embodiments, the mRNA encodes a cytokine. In some embodiments, the cytokine is granulocyte-macrophage colony-stimulating factor (GM-CSF).

In some embodiments, the siRNA is a SIRT1 siRNA, e.g., according to SEQ ID NO: 2.

In some embodiments, the nucleic acid is DNA. In some embodiments, the DNA is plasmid DNA.

In some embodiments, the nucleic acid is double stranded. In some embodiments, the nucleic acid is single stranded. In some embodiments, the nucleic acid is a dinucleotide (e.g., is 2 nucleotides in length). In some embodiments, the nucleic acid is at least two nucleotides long. In some embodiments, the nucleic acid comprises CpG oligodeoxynucleotides. In some embodiments, the nucleic acid is unmethylated. In some embodiments, the nucleic acid is methylated.

In some embodiments, the nucleic acid is about 15, 16, 17, 18, 19, 20, 21, 22, 23, 24, 25, 26, 27, 28, 29, or 30 nucleotides in length. In some embodiments, the nucleic acid is about 15-20, 20-25, 25-30, or 15-30 nucleotides in length. In some embodiments, the nucleic acid is about 100, 200, 300, 400, 500, 600, 700, 800, 900, 1000, 1250, 1500, 1750, 2000, or 2500 nucleotides in length. In some embodiments, the nucleic acid is 100-500, 500-1000, 1000-1500, 1500-2000, 2000-2500, or 100-2500 nucleotides in length.

### Small Molecules

In some embodiments, the NPs comprise a small molecule. In some embodiments, the small molecule is within the nanoparticle. In some embodiments, the small molecule is soluble in aqueous solutions. In some embodiments, the small molecule is soluble in organic solutions. Exemplary small molecule effectors include trametinib and doxorubicin.

### Trametinib

In some embodiments, the small molecule is a kinase inhibitor, e.g., a MEK inhibitor. In some embodiments, the MEK inhibitor is trametinib. In some embodiments, trametinib comprises the structure according to Formula VI:

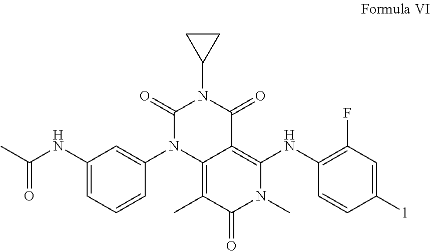

### Doxorubicin

In some embodiments, the small molecule is a chemotherapeutic. In some embodiments, the small molecule intercalates with nucleic acids, e.g., DNA. In some embodiments, the small molecule that intercalates with nucleic acids is doxorubicin. In some embodiments, doxorubicin comprises the structure according to Formula VII:

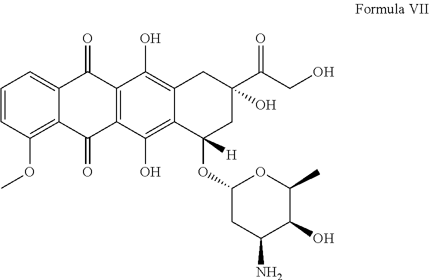 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

### Methods of Making

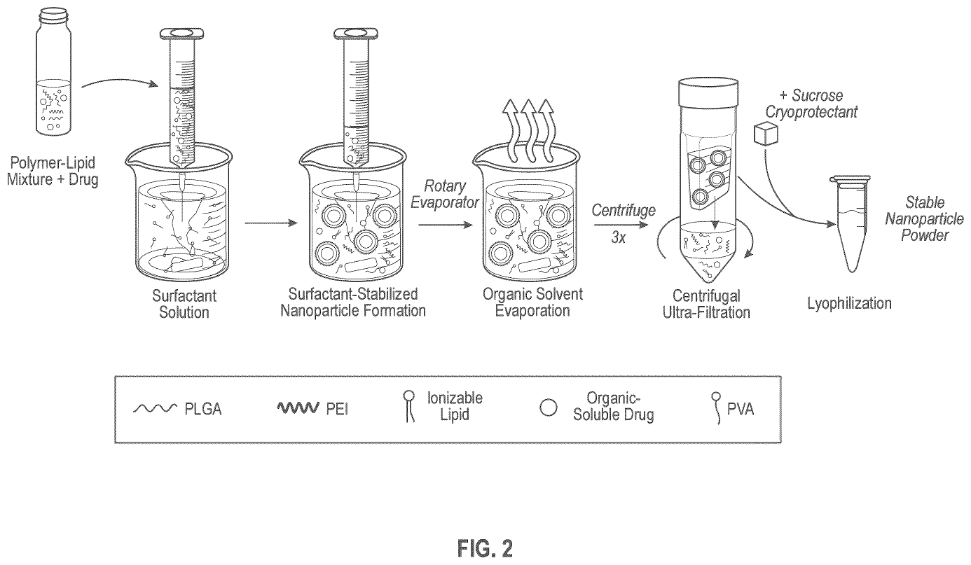

Nanoparticles may be made as described herein, e.g., in Example 1. In some embodiments, nanoparticles are synthesized using nanoprecipitation (e.g., as shown in FIG. 2). In some embodiments, certain components of the nanoparticles (e.g., a non-cationic polymer, a cationic polymer, and an ionizable lipid) are mixed in an organic phase, e.g., in containing acetone. In some embodiments, certain components of the nanoparticles (e.g., a cationic polymer and an ionizable lipid) are added at the aqueous phase, e.g., containing polyvinyl alcohol (PVA) or Vitamin E polyethylene glycol succinate (TPGS). In some embodiments, a non-cationic polymer (e.g., PLGA) is dissolved in an organic phase, e.g., acetone. In some embodiments, a cationic polymer (e.g., PEI) is added to the non-cationic polymer in the organic phase. In some embodiments, an ionizable lipid (e.g., MC3) is added to the organic phase. In some embodiments, small molecules, e.g., doxorubicin or trametinib, are added to the organic phase.

In some embodiments, the organic phase is added to an aqueous phase (e.g., containing PVA or TPGS), e.g., using a syringe, resulting in a mixed phase solution. In some embodiments, the organic solvent is evaporated from the mixed phase solution.

In some embodiments, nanoparticles are made using the formulations shown in Table E1. In some embodiments, nanoparticles are made using the formulations shown in Table 1, Table 2, or Table 3.

TABLE 1
Exemplary nanoparticle formulations

| | | | | | | AQUEOUS PHASE (in the beaker) | | | | | | |
|---|---|---|---|---|---|---|---|---|---|---|---|---|
| | ORGANIC PHASE (in the syringe) | | | | | | | PVA | | | | |
| | PLGA | Acetone | PAMAM | PEI | Lipid | Lipid | PEI | (% w/v, | | Size | Charge | |
| NP # | (mg) | (mL) | (mg) | (mg) | (μL) | (μL) | (mg) | mL) | RPM | (d · nm) | (mV) | Notes |
| 1A | 50 | 5 | 6.25 | x | x | x | x | 0.10% | 600 | 253 | 64 | |
| | | | | | | | | 15 | | | | |
| 2A | 50 | 5 | 6.25 | x | x | x | x | 0.10% | 1000 | 237 | 56 | |
| | | | | | | | | 15 | | | | |
| 3A | 25 | 5 | 2.5 | x | x | x | x | 0.10% | 1000 | 165 | 27 | |
| | | | | | | | | 15 | | | | |
| 4A | 25 | 7 | 2.5 | x | x | x | x | 0.10% | 1000 | 156 | 33 | |
| | | | | | | | | 15 | | | | |
| 5A | 25 | 7 | 2.5 | x | 1 | x | x | 0.10% | 1000 | 173 | 37 | |
| | | | | | MC3 | | | 15 | | | | |
| 6A | 25 | 7 | 2.5 | x | x | x | x | 0.20% | 1000 | 161 | 29 | |
| | | | | | | | | 15 | | | | |
| 7A | 25 | 7 | 1.25 | x | x | x | x | 0.10% | 1000 | 116 | 42 | |
| | | | | | | | | 15 | | | | |
| 8A | 25 | 7 | x | x | 2 | x | x | 0.10% | 1000 | x | x | Precipitated |
| | | | | | MC3 | | | 15 | | | | |
| 9A | 25 | 7 | x | x | 2 | x | x | 0.10% | 1000 | x | x | MC3 is dissolved in methanol, |
| | | | | | MC3 | | | 15 | | | | Precipitated |
| 10A | 25 | 7 | x | x | 1 | x | x | 0.10% | 1000 | x | x | Precipitated |
| | | | | | MC3 | | | 15 | | | | |
| 11A | 25 | 7 | x | x | 1 | x | x | 0.10% | 1000 | 194 | −2.1 | Heated to 40° C., Precipitated |
| | | | | | MC3 | | | 15 | | | | |
| 12A | 25 | 2 | x | x | 1 | x | x | 0.10% | 1000 | 157 | −9 | Heated to 50° C., Precipitated |
| | | | | | MC3 | | | 15 | | | | |
| 13A | 25 | 2 | x | x | 1 | x | x | 0.20% | 1000 | x | x | Heated to 50° C., Precipitated |
| | | | | | MC3 | | | 15 | | | | |
| 14A | 25 | 2 | 0.05 | x | 1 | x | x | 0.10% | 1000 | x | x | Precipitated |
| | | | | | MC3 | | | 15 | | | | |
| 15A | 25 | 7 | 0.5 | x | 1 | x | x | 0.10% | 1000 | x | x | Precipitated |
| | | | | | MC3 | | | 15 | | | | |
| 16A | 25 | 2 | 0.5 | x | 1 | x | x | 0.10% | 1000 | 2147 | −2.8 | |
| | | | | | MC3 | | | 15 | | | | |
| 17A | 25 | 7 | 0.5 | x | 1 | x | x | 0.20% | 1000 | 331 | −7.7 | |
| | | | | | MC3 | | | 15 | | | | |
| 18A | 25 | 7 | 0.5 | x | 1 | x | x | 0.10% | 1000 | 239 | 24 | TPGS used instead of PVA |
| | | | | | MC3 | | | 15 | | | | |
| 19A | 25 | 7 | 0.5 | x | x | x | x | 0.10% | 1000 | 341 | 28 | TPGS used instead of PVA |
| | | | | | | | | 15 | | | | |
| 20A | 25 | 7 | 5 | x | X | x | x | 0.10% | 1000 | 260 | 64 | TPGS used instead of PVA |
| | | | | | | | | 15 | | | | |
| 21A | 25 | 7 | 0.5 | x | X | x | x | 0.10% | 1000 | 274 | 39 | 190 μL methanol used to |
| | | | | | | | | 15 | | | | dissolve PAMAM |
| 22A | 25 | 7 | 0.5 | x | x | x | x | 0.10% | 1000 | 277 | 45 | |
| | | | | | | | | 15 | | | | |
| 23A | 25 | 7 | 1.25 | x | x | x | x | 1.00% | 1000 | 112 | 50 | 1.5 mL 1% PVA + 100 μL 1% |
| | | | | | | | | 15 | | | | TPGS |
| 24A | 25 | 7 | 1.25 | x | x | x | x | 1.00% | 1000 | 113 | 59 | 0.75 mL 1% PVA + 0.75 mL |
| | | | | | | | | 15 | | | | 1% TPGS |
| 25A | 25 | 7 | 1.25 | x | 1 | x | x | 0.10% | 1000 | 176 | 73 | TPGS used instead of PVA |
| | | | | | MC3 | | | 15 | | | | |
| 26A | 25 | 7 | 1.25 | x | x | 1 | x | 0.10% | 1000 | 172 | 65 | TPGS used instead of PVA |
| | | | | | | MC3 | | 15 | | | | Aqueous Phase replaced by |
| | | | | | | | | | | | | Ethanol |
| 27A | 25 | 7 | 1.25 | x | x | 1 | x | 0.10% | 1000 | x | x | Precipitated |
| | | | | | | MC3 | | 15 | | | | Aqueous Phase replaced by |
| | | | | | | | | | | | | Ethanol |
| 28A | 25 | 7 | 0.75 | x | x | | x | 0.10% | 1000 | 153 | 52 | TPGS used instead of PVA |
| | | | | | | | | 15 | | | | |
| 29A | 25 | 7 | x | x | 1 | x | x | 0.10% | 1000 | 162 | 26 | TPGS used instead of PVA |
| | | | | | MC3 | | | 15 | | | | |
| 30A | 25 | 7 | x | x | 1 | x | x | 0.50% | 1000 | 95 | −9.2 | TPGS used instead of PVA |
| | | | | | MC3 | | | 15 | | | | |
| 31A | 25 | 7 | 0.25 | x | 1 | x | x | 0.50% | 1000 | 257 | 11 | TPGS used instead of PVA |
| | | | | | MC3 | | | 15 | | | | |
| 32A | 25 | 7 | x | 3 | x | x | x | 0.10% | 1000 | 151 | 55 | |
| | | | | | | | | 15 | | | | |
| 33A | 25 | 7 | x | 3 | x | x | 3 | 0.10% | 1000 | 226 | 42 | |
| | | | | | | | | 15 | | | | |
| 34A | 25 | 7 | x | x | x | x | 3 | 0.50% | 1000 | 133 | 43 | |
| | | | | | | | | 15 | | | | |
| 35A | 25 | 7 | x | x | x | x | 0.6 | 0.10% | 1000 | 178 | 48 | |
| | | | | | | | | 15 | | | | |
| 36A | 15 | 7 | x | x | x | x | 3 | 0.50% | 1000 | 130 | 40 | |
| | | | | | | | | 15 | | | | |

TABLE 2
Exemplary Nanoparticle Formulations

| | | | | | | AQUEOUS PHASE (in the beaker) | | | | | | |
|---|---|---|---|---|---|---|---|---|---|---|---|---|
| | ORGANIC PHASE (in the syringe) | | | | | | | PVA | | | | |
| | PLGA | Acetone | PAMAM | PEI | Lipid | Lipid | PEI | (% w/v, | | Size | Charge | |
| NP # | (mg) | (mL) | (mg) | (mg) | (μL) | (μL) | (mg) | mL) | RPM | (d · nm) | (mV) | Notes |
| 37A | 15 | 7 | x | x | x | x | 3 | 0.50% | 1000 | 205 | 60 | TPGS |
| | | | | | | | | 15 | | | | |
| 38A | 15 | 7 | x | x | 1 | x | 3 | 0.50% | 1000 | 164 | 62 | TPGS |
| | | | | | MC3 | | | 15 | | | | |
| 39A | 15 | 7 | x | x | x | x | 1 | 0.50% | 1000 | 122 | 46 | 7.4 mL of 1% PVA + 100 μL |
| | | | | | | | | 15 | | | | of 1% TPGS |
| 40A | 15 | 7 | x | x | 1 | x | 3 | 0.50% | 1000 | 130 | 40 | 7.4 mL PVA + 100 μL TPGS |
| | | | | | MC3 | | | 15 | | | | |
| 41A | 15 | 7 | x | x | 1 | x | 3 | 0.50% | 1000 | 148 ± 5 | 43 ± 4 | |
| | | | | | MC3 | | | 15 | | | | |
| 42A | 15 | 7 | x | x | 1 | x | 3 | 0.50% | 1000 | 150 ± 6 | 46 ± 1 | |
| | | | | | SM | | | 15 | | | | |
| 43A | 15 | 7 | x | x | 1 | x | 3 | 0.50% | 1000 | 164 ± 36 | 45 ± 4 | |
| | | | | | ALC | | | 15 | | | | |
| 44A | 15 | 7 | x | x | 1 | x | 3 | 0.50% | 1000 | 173 | 47 | 7 mL of 1% PVA + 500 μL of |
| | | | | | MC3 | | | 15 | | | | 1% TPGS |
| 45A | 15 | 7 | x | x | 1 | x | 3 | 0.50% | 1000 | 144 | 47 | 5 mL of 1% PVA + 2.5 mL of |
| | | | | | MC3 | | | 15 | | | | 1% TPGS |
| 46A | 15 | 7 | x | x | 1 | x | 3 | 0.50% | 1000 | 161 | 47 | 3.75 mL of 1% PVA + 3.75 |
| | | | | | MC3 | | | 15 | | | | mL of 1% TPGS |
| 47A | 15 | 7 | x | x | 2 | x | 3 | 0.50% | 1000 | 148 | 42 | |
| | | | | | MC3 | | | 15 | | | | |
| 48A | 15 | 7 | x | x | 3 | x | 3 | 0.50% | 1000 | 183 | 46 | |
| | | | | | MC3 | | | 15 | | | | |
| 49A | 15 | 7 | x | x | 1 | x | 2 | 0.50% | 1000 | 151 ± 18 | 43 ± 2 | |
| | | | | | MC3 | | | 15 | | | | |
| 50A | 15 | 7 | x | x | 1 | x | 1 | 0.50% | 1000 | 149 | 44 | |
| | | | | | MC3 | | | 15 | | | | |
| 51A | 15 | 7 | x | x | 1 | x | 0.5 | 0.50% | 1000 | 140 | 44 | |
| | | | | | MC3 | | | 15 | | | | |
| 52A | 15 | 7 | x | 3 | 1 | x | x | 0.50% | 1000 | 141 ± 9 | 54 ± 4 | |
| | | | | | MC3 | | | 15 | | | | |
| 53A | 15 | 7 | x | x | 2 | x | 1 | 0.50% | 1000 | 162 | 41 | |
| | | | | | MC3 | | | 15 | | | | |
| 54A | 15 | 7 | x | 3 | 2 | x | x | 0.50% | 1000 | 149 | 45 | |
| | | | | | MC3 | | | 15 | | | | |
| 55A | 15 | 7 | x | 3 | 3 | x | x | 0.50% | 1000 | 177 | 43 | |
| | | | | | MC3 | | | 15 | | | | |
| 56A | 15 | 7 | x | 3 | 1 | x | x | 0.50% | 1000 | 144 | 45 | |
| | | | | | SM | | | 15 | | | | |
| 57A | 15 | 7 | x | 3 | 1 | x | x | 0.50% | 1000 | 139 | 44 | |
| | | | | | ALC | | | 15 | | | | |
| 58A | 15 | 7 | x | 1.5 | 2 | x | x | 0.50% | 1000 | 152 ± 7 | 52 ± 4 | |
| | | | | | MC3 | | | 15 | | | | |
| 59A | 15 | 7 | x | 3 | 1 | x | x | 0.50% | 1000 | 134 | 51 | |
| | | | | | MC3 | | | 15 | | | | |
| 60A | 15 | 7 | x | 1.5 | 2 | x | x | 0.50% | 1000 | 159 | 48 | |
| | | | | | MC3 | | | 15 | | | | |
| 61A | 15 | 7 | x | 1 | 3 | x | x | 0.50% | 1000 | 171 | 46 | |
| | | | | | MC3 | | | 15 | | | | |
| 62A | 15 | 7 | x | 0.5 | 3 | x | x | 0.50% | 1000 | 134 | 38 | |
| | | | | | MC3 | | | 15 | | | | |
| 63A | 15 | 7 | x | 1.5 | 2 | x | x | 0.50% | 1000 | 178 | 52 | |
| | | | | | MC3 | | | 15 | | | | |
| 64A | 15 | 7 | x | 1.5 | 2 | x | x | 0.50% | 1000 | 124 | 49 | |
| | | | | | ALC | | | 15 | | | | |
| 65A | 15 | 7 | x | 1.5 | 2 | x | x | 0.50% | 1000 | 125 | 53 | |
| | | | | | SM | | | 15 | | | | |
| 66A | 15 | 7 | x | 1.5 | 2 | x | x | 0.50% | 1000 | 146 ± 5 | 53 ± 1 | SUN_{DP} |
| | | | | | MC3 | | | 15 | | | | |
| 67A | 15 | 7 | x | 1.5 | 2 | x | x | 0.50% | 1000 | | | Linear PEI (Mn 10,000) was |
| | | | | | MC3 | | | 15 | | | | substituted for branched PEI |
| 68A | 15 | 7 | x | 1.5 | 2 | x | x | 0.50% | 1000 | | | PLGA was replaced by |
| | | | | | MC3 | | | 15 | | | | PLGA-PEG |

TABLE 3
Exemplary Nanoparticle Formulations

| | | | | | | AQUEOUS PHASE | | | | | | |
|---|---|---|---|---|---|---|---|---|---|---|---|---|
| | ORGANIC PHASE | | | | | | | PVA | | | | |
| | PLGA | Acetone | DMF | PEI | Lipid | Lipid | PEI | (% w/v, | | | Size | OTHER / |
| NP # | (mg) | (mL) | (mL) | (mg) | (μL) | (μL) | (mg) | mL) | RPM | AdditionMethod | (d · nm) | NOTES |

| | | | | | | | | | | | |
|---|---|---|---|---|---|---|---|---|---|---|---|
| 69A | 15 | 7 | 0.1 | 1.5 | x | x | x | 0.5% | 1000 | Dropwise | 148 |
| | | | | | | | | 15 | | | |
| 70A | 15 | 7 | 0.1 | 1.5 | x | x | x | 0.1% | 1000 | Injection | 78 |
| | | | | | | | | 15 | | | |
| 71A | 15 | 7 | x | 1.5 | x | x | x | 0.1% | 1000 | Injection | 79 |
| | | | | | | | | 15 | | | |
| 72A | 15 | 7 | 0.2 | 1.5 | x | x | x | 0.1% | 1000 | Injection | 84 |
| | | | | | | | | 15 | | | |
| 73A | 15 | 5.5 | x | 1.5 | x | x | x | 0.2% | 1000 | Injection | 84 |
| | | | | | | | | 15 | | | |
| 74A | 15 | 3.5 | x | 1.5 | x | x | x | 0.1% | 1000 | Injection | 94 |
| | | | | | | | | 15 | | | |
| 75A | 15 | 7 | x | 1.5 | x | x | x | 0.1% | 1000 | Pump | 118 |
| | | | | | | | | 15 | | 5 mL/min | |
| 76A | 15 | 7 | x | 1.5 | x | x | x | 0.1% | 1000 | Pump | 143 |
| | | | | | | | | 15 | | 2.5 mL/min | |
| 77A | 15 | 7 | 0.5 | 1.5 | x | x | x | 0.1% | 1000 | Dropwise | 76 |
| | | | | | | | | 15 | | | |
| 78A | 15 | 7 | 0.25 | 1.5 | x | x | x | 0.1% | 1000 | Dropwise | 122 |
| | | | | | | | | 15 | | | |

In some embodiments, the nanoparticles are dried, e.g., using lyophilization. In some embodiments lyophilization is achieved by a low temperature dehydration process that involves freezing the NPs and lowering pressure, thereby removing ice by sublimation. In some embodiments, the NPs are lyophilized in the presence of antioxidants. In some embodiments, sucrose is added to the nanoparticle solution prior to lyophilization. In some embodiments, transfection efficiency is not reduced after lyophilization.

### Methods of Delivery

Prior to delivery, the NPs may be provided in various suitable forms. In some embodiments, the NPs are powder (e.g., lyophilized). In some embodiments, the NPs are packaged with a nucleic acid (e.g., mRNA, siRNA, or pDNA). In some embodiments, the nucleic acid is powder (e.g., lyophilized). In some embodiments, the nucleic acid is not preloaded in the delivery system. In some embodiments, the lyophilized NPs and nucleic acid are mixed with an aqueous solution (e.g., water) before administration (e.g., injection, e.g., intramuscular, intravenous, or subcutaneous injection). In some embodiments, complexation of the NPs and the nucleic acid is achieved by mixing (e.g., shaking by hand). In some embodiments, complexation of the NPs and the nucleic acid is instantaneous. In some embodiments, the NPs and the nucleic acid are in the same container. In some embodiments, the NPs are in a first container and the nucleic acid is in a different container.

In some embodiments, the nucleic acid is in an aqueous solution (e.g., water or a buffer). In some embodiments, the powdered NPs are loaded separately from the liquid nucleic acid, e.g., in a dual chambered vessel (e.g., syringe) comprising a breakable septum. In some embodiments, the septum between the lyophilized NPs and the nucleic acid is broken prior to administration (e.g., less than 1 hour, 30 minutes, 10 minutes, or 5 minutes prior to administration). In some embodiments, the NPs are administered by injection, e.g., intramuscular, intravenous, or subcutaneous injection.

### Methods of Use

In some embodiments, the compositions described herein can be used to deliver therapeutic effectors to a cell or a subject. In some embodiments, the compositions described herein can be used in the treatment or prevention of cancer, e.g., by as a cancer vaccine. In some embodiments, the compositions described herein can be used to deliver mRNA encoding for a tumor antigen, e.g., from a patient's own tumor. In some embodiments, the compositions described herein can be delivered intratumorally, e.g., by injection. In some embodiments, the cancer is melanoma.

In some embodiments, the compositions described herein can be used to vaccinate or induce an immune response against a particular antigen, e.g., by delivering mRNA encoding for an immunogenic protein or antigen.

### Kits

In some embodiments, the reagents or components described herein may be included in a kit. In some embodiments, the kit comprises one or more of the reagents or components described herein. In some embodiments, the kit comprises a package insert or other labeling including instructions for performing an assay as described herein. In some embodiments, the kit comprises a container.

### EXAMPLES

### Example 1: Exemplary Nanoparticle Formulations

This example describes the production of 71 batches of nanoparticles with exemplary compositions and synthesis methods (Table E1). Formulations of Tables 1, 2, and 3 were also tested. Poly(lactic-co-glycolic acid) (PLGA) was the core polymer. Two different cationic polymers, poly(amidoamine) (PAMAM) and polyethyleneimine (PEI), and three different commercially available ionizable lipids, SM-102 (SM); ALC-0315 (ALC) and D-Lin-MC3-DMA (MC3) were tested in different formulations and synthesis methods. Exemplary nanoparticle formulations were also complexed with GFP mRNA.

### Nanoparticle Synthesis

Resomer RG 504 H poly(D,L-lactide-co-glycolide) 50:50 with a molecular weight of 38-54 kDa was purchased from Sigma-Aldrich (cat. 719900). Branched polyethyleneimine with an average molecular weight of 25 kDa was purchased from Sigma-Aldrich (cat. 408727). 5th generation poly(amidoamine) dendrimer was purchased from Sigma-Aldrich (cat. 536709). Ionizable lipids were purchased from BroadPharma: D-Lin-MC3-DMA (MC3) (cat. BP-25497), SM-102 (SM) (cat. BP-25499), and ALC-0315 (ALC) (cat. BP-25498). These were made into stock solutions of 1 g/mL in ethanol and stored at −20° C. until use. 80% hydrolyzed polyvinyl alcohol (PVA) with a molecular weight of 9-10 kDa was purchased from Sigma-Aldrich (cat. 360627) and made into stock solutions in ultrapure water (ResinTech CLIR 5000 series). D-α-Tocopherol polyethylene glycol 1000 succinate (TPGS) was purchased from Sigma-Aldrich (cat. 57668) and made into stock solutions in ultrapure water. Doxorubicin free base was purchased from ApexBio (cat. A3966) and trametinib was purchased from MedChem Express (cat. HY-10999). Both drugs were stored at −20° C. and protected from light until use.

All NPs were synthesized using nanoprecipitation as shown in FIG. 2. The NP hydrodynamic diameter, polydispersity index, zeta potential, transfection capability and safety, and reproducibility of the method were tested. The synthesis method of exemplary NP 71 is described as follows: First, 15 mg of PLGA was dissolved in 7 mL of acetone and PEI was prepared as a 3 mg/mL solution in acetone. From stock solutions of ionizable lipids, 2 μL were taken and dissolved in 100 μL of ethanol. All polymer and lipid solutions were allowed to dissolve in a temperature-controlled ultrasonic bath for 30 minutes in the case of polymers and 5 minutes in the case of lipids. To create the organic phase for nanoprecipitation, 1.5 mg of PEI (0.5 mL) was added to the PLGA solution and vortexed. Next, all of the lipid solution in ethanol was added and vortexed. Therefore, the final organic phase composition consisted of 15 mg PLGA, 1.5 mg PEI, and 2 mg of ionizable lipids. To make drug-loaded nanoparticles, doxorubicin or trametinib were dissolved in DMSO to create highly concentrated stock solutions (usually between 10-500 mg/mL) and 10 μL of these solutions were added to the organic phase. At such a small volume, the presence of DMSO had no effect on the morphology or performance of the drug-loaded NPs.

The aqueous phase of 15 mL of 0.5% PVA was added to a beaker containing a stir bar and placed on a magnetic stir plate at 1000 rpm and room temperature. A syringe with a 27 G needle was clamped to the stir plate and lowered until the needle was inside of the vortex just above the stir bar. The organic phase was added to the syringe and dripping of the organic phase was initiated by flushing the needle port with organic phase solution. After around 1 hour of passively dripping, all of the organic phase was added to the aqueous phase.

Afterwards, the mixed phase solution was added to an aluminum foil-covered round bottom flask and the organic solvent was evaporated using a rotary evaporator (BUCHI, Rotavapor R-300) set to 30 mbar and rotation speed of 30 rpm. After 20-30 minutes, all of the organic solvent was removed. Next, the leftover solution was added to a centrifugal concentrator with a 300 kDa filter (Fisher Scientific, cat. 14-558-511) and centrifuged in a bucket rotor at 2000 g for 1 hour to remove any unreacted components. The bottom chamber solution was discarded and the top chamber solution containing NPs topped up to 10 mL using ultrapure water. NPs were centrifuged again, the bottom chamber solution was discarded, and the top chamber solution topped up to 10 mL. NPs were centrifuged once more, the bottom chamber solution discarded, and the top chamber solution collected. After all 3 centrifugations, around 300 μL of NP solution was collected. This solution was mixed in a 1:1 volume ratio with 100 mg/mL sucrose (Sigma-Aldrich, cat. S0389) in ultrapure water. Sucrose may act as a cryoprotectant, for example, by lessening the aggregation of NPs during lyophilization. 20 μL volumes of NP-sucrose solution were aliquoted into 0.5 mL centrifuge tubes (MTC Bio, cat. C2007) and covered with aluminum foil poked with holes. These tubes were placed into aluminum foil-covered freeze-drying flasks and lyophilized overnight for a maximum of 24 hours. NP powders were stored at −20° C.

TABLE E1
Exemplary nanoparticle formulations

| | | | | | | AQUEOUS PHASE | | | | | | |
|---|---|---|---|---|---|---|---|---|---|---|---|---|
| | ORGANIC PHASE | | | | | | | PVA | | | | |
| | PLGA | Acetone | PAMAM | PEI | Lipid | Lipid | PEI | (% w/v, | | Size | Charge | |
| NP # | (mg) | (mL) | (mg) | (mg) | (μL) | (μL) | (mg) | mL) | RPM | (d · nm) | (mV) | |
| 1 | 50 | 5 | 6.25 | x | x | x | x | 0.1% | 600 | 253 | 64 | |
| | | | | | | | | 15 | | | | |
| 2 | 50 | 5 | 6.25 | x | x | x | x | 0.1% | 1000 | 237 | 56 | |
| | | | | | | | | 15 | | | | |
| 3 | 25 | 5 | 2.5 | x | x | x | x | 0.1% | 1000 | 165 | 27 | |
| | | | | | | | | 15 | | | | |
| 4 | 25 | 7 | 2.5 | x | x | x | x | 0.1% | 1000 | 156 | 33 | |
| | | | | | | | | 15 | | | | |
| 5 | 25 | 7 | 2.5 | x | 1 | x | x | 0.1% | 1000 | 173 | 37 | |
| | | | | | MC3 | | | 15 | | | | |
| 6 | 25 | 7 | 2.5 | x | X | x | x | 0.2% | 1000 | 161 | 29 | |
| | | | | | | | | 15 | | | | |
| 7 | 25 | 7 | 1.25 | x | X | x | x | 0.1% | 1000 | 116 | 42 | |
| | | | | | | | | 15 | | | | |
| 8 | 25 | 7 | X | x | 2 | x | x | 0.1% | 1000 | X | X | Precipitated |
| | | | | | MC3 | | | 15 | | | | |
| 9 | 25 | 7 | X | x | 2 | x | x | 0.1% | 1000 | X | X | MC3 in Methanol, |
| | | | | | MC3 | | | 15 | | | | Precipitated |
| 10 | 25 | 7 | X | x | 1 | x | x | 0.1% | 1000 | X | X | Precipitated |
| | | | | | MC3 | | | 15 | | | | |
| 11 | 25 | 7 | X | x | 1 | x | x | 0.1% | 1000 | 194 | −2.1 | Heated to 40° C., |
| | | | | | MC3 | | | 15 | | | | Precipitated |
| 12 | 25 | 2 | X | x | 1 | x | x | 0.1% | 1000 | 157 | −9.0 | Heated to 50° C., |
| | | | | | MC3 | | | 15 | | | | Precipitated |
| 13 | 25 | 2 | X | x | 1 | x | x | 0.2% | 1000 | X | X | Heated to 50° C., |
| | | | | | MC3 | | | 15 | | | | Precipitated |
| 14 | 25 | 2 | 0.05 | x | 1 | x | x | 0.1% | 1000 | X | X | Precipitated |
| | | | | | MC3 | | | 15 | | | | |
| 14-2 | 25 | 2 | 0.05 | x | 1 | x | x | 0.1% | 1000 | X | X | Precipitated |
| | | | | | MC3 | | | 15 | | | | |
| 15 | 25 | 7 | 0.5 | x | 1 | x | x | 0.1% | 1000 | X | X | Precipitated |
| | | | | | MC3 | | | 15 | | | | |
| 16 | 25 | 7 | 0.5 | x | 1 | x | x | 0.1% | 1000 | 2147 | −2.8 | |
| | | | | | MC3 | | | 15 | | | | |
| 17 | 25 | 7 | 0.5 | x | 1 | x | x | 0.2% | 1000 | 331 | −7.7 | |
| | | | | | MC3 | | | 15 | | | | |
| 18 | 25 | 7 | 0.5 | x | 1 | x | x | 0.1% | 1000 | 239 | 24 | TPGS used instead |
| | | | | | MC3 | | | 15 | | | | of PVA |
| 19 | 25 | 7 | 0.5 | x | x | x | x | 0.1% | 1000 | 341 | 28 | TPGS |
| | | | | | | | | 15 | | | | |
| 20 | 25 | 7 | 5 | x | x | x | x | 0.1% | 1000 | 260 | 64 | TPGS |
| | | | | | | | | 15 | | | | |
| 21 | 25 | 7 | 0.5 | x | x | x | x | 0.1% | 1000 | 274 | 39 | 190 μL methanol |
| | | | | | | | | 15 | | | | used to dissolve |
| | | | | | | | | | | | | PAMAM |
| 22 | 25 | 7 | 0.5 | x | x | x | x | 0.1% | 1000 | 277 | 45 | |
| | | | | | | | | 15 | | | | |
| 23 | 25 | 7 | 1.25 | x | x | x | x | 1.0% | 1000 | 112 | 50 | 1.5 mL 1% PVA + |
| | | | | | | | | 15 | | | | 100 μL 1% TPGS |
| 24 | 25 | 7 | 1.25 | x | x | x | x | 1.0% | 1000 | 113 | 59 | 0.75 mL 1% PVA + |
| | | | | | | | | 15 | | | | 0.75 mL 1% TPGS |
| 25 | 25 | 7 | 1.25 | x | 1 | x | x | 0.1% | 1000 | 176 | 73 | TPGS |
| | | | | | MC3 | | | 15 | | | | |
| 26 | 25 | 7 | 1.25 | x | x | 1 | x | 0.1% | 1000 | 172 | 65 | TPGS, Aqueous |
| | | | | | | MC3 | | 15 | | | | Phase is Ethanol |
| 27 | 25 | 7 | 1.25 | x | x | 1 | x | 0.1% | 1000 | x | x | Precipitated, |
| | | | | | | MC3 | | 15 | | | | Aqueous Phase is |
| | | | | | | | | | | | | Ethanol |
| 28 | 25 | 7 | 0.75 | x | x | | x | 0.1% | 1000 | 153 | 52 | TPGS |
| | | | | | | | | 15 | | | | |
| 29 | 25 | 7 | x | x | 1 | x | x | 0.1% | 1000 | 162 | 26 | TPGS |
| | | | | | MC3 | | | 15 | | | | |
| 30 | 25 | 7 | x | x | 1 | x | x | 0.5% | 1000 | 95 | −9.2 | TPGS |
| | | | | | MC3 | | | 15 | | | | |
| 31 | 25 | 7 | 0.25 | x | 1 | x | x | 0.5% | 1000 | 257 | 11 | TPGS |
| | | | | | MC3 | | | 15 | | | | |
| 32 | 25 | 7 | x | 3 | x | x | x | 0.1% | 1000 | 151 | 55 | |
| | | | | | | | | 15 | | | | |
| 33 | 25 | 7 | x | 3 | x | x | 3 | 0.1% | 1000 | 226 | 42 | |
| | | | | | | | | 15 | | | | |
| 34 | 25 | 7 | x | x | x | x | 3 | 0.5% | 1000 | 133 | 43 | |
| | | | | | | | | 15 | | | | |
| 35 | 25 | 7 | x | x | x | x | 0.6 | 0.1% | 1000 | 178 | 48 | |
| | | | | | | | | 15 | | | | |
| 36 | 15 | 7 | x | x | x | x | 3 | 0.5% | 1000 | 130 | 40 | |
| | | | | | | | | 15 | | | | |
| 37 | 15 | 7 | x | x | x | x | 1 | 0.5% | 1000 | 205 | 60 | TPGS |
| | | | | | | | | 15 | | | | |
| 38 | 15 | 7 | x | x | x | 1 | 1 | 0.5% | 1000 | 164 | 62 | TPGS, Aqueous |
| | | | | | | MC3 | | 15 | | | | Phase is Ethanol |
| 39 | 15 | 7 | x | x | x | X | 1 | 0.5% | 1000 | 122 | 46 | 7.4 mL PVA + 100 |
| | | | | | | | | 15 | | | | μL TPGS |
| 40 | 15 | 7 | x | x | x | 1 | 1 | 0.5% | 1000 | 130 | 40 | 7.4 mL PVA + 100 |
| | | | | | | MC3 | | 15 | | | | μL TPGS, Aqueous |
| | | | | | | | | | | | | Phase is Ethanol |
| 41 | 15 | 7 | x | x | x | 1 | 3 | 0.5% | 1000 | 147 | 43 | Aqueous Phase is |
| | | | | | | MC3 | | 15 | | | | Ethanol |
| 42 | 15 | 7 | x | x | x | 1 | 3 | 0.5% | 1000 | 145 | 45 | Aqueous Phase is |
| | | | | | | SM | | 15 | | | | Ethanol |
| 43 | 15 | 7 | x | x | x | 1 | 3 | 0.5% | 1000 | 189 | 47 | Aqueous Phase is |
| | | | | | | ALC | | 15 | | | | Ethanol |
| 44 | 15 | 7 | x | x | x | 3 | 3 | 0.5% | 1000 | 173 | 47 | 7 mL PVA + 500 |
| | | | | | | MC3 | | 15 | | | | μL TPGS, Aqueous |
| | | | | | | | | | | | | Phase is Ethanol |
| 45 | 15 | 7 | x | x | x | 3 | 3 | 0.5% | 1000 | 144 | 47 | 5 mL PVA + 2.5 |
| | | | | | | MC3 | | 15 | | | | mL TPGS, Aqueous |
| | | | | | | | | | | | | Phase is Ethanol |
| 46 | 15 | 7 | x | x | x | 3 | 3 | 0.5% | 1000 | 161 | 47 | 3.75 mL PVA + 3.75 |
| | | | | | | MC3 | | 15 | | | | mL TPGS, Aqueous |
| | | | | | | | | | | | | Phase is Ethanol |
| 47 | 15 | 7 | x | x | 1 | x | 3 | 0.5% | 1000 | 144 | 47 | |
| | | | | | MC3 | | | 15 | | | | |
| 48 | 15 | 7 | x | x | 2 | x | 3 | 0.5% | 1000 | 148 | 42 | |
| | | | | | MC3 | | | 15 | | | | |
| 49 | 15 | 7 | x | x | 3 | x | 3 | 0.5% | 1000 | 183 | 46 | |
| | | | | | MC3 | | | 15 | | | | |
| 50 | 15 | 7 | x | x | 1 | x | 2 | 0.5% | 1000 | 138 | 44 | |
| | | | | | MC3 | | | 15 | | | | |
| 51 | 15 | 7 | x | x | 1 | x | 1 | 0.5% | 1000 | 149 | 44 | |
| | | | | | MC3 | | | 15 | | | | |
| 52 | 15 | 7 | x | x | 1 | x | 0.5 | 0.5% | 1000 | 140 | 44 | |
| | | | | | MC3 | | | 15 | | | | |
| 53 | 15 | 7 | x | x | 1 | x | 3 | 0.5% | 1000 | 154 | 47 | |
| | | | | | SM | | | 15 | | | | |
| 54 | 15 | 7 | x | x | 1 | x | 3 | 0.5% | 1000 | 138 | 42 | |
| | | | | | ALC | | | 15 | | | | |
| 55 | 15 | 7 | x | x | 1 | x | 3 | 0.5% | 1000 | 154 | 40 | |
| | | | | | MC3 | | | 15 | | | | |
| 56 | 15 | 7 | x | 3 | 1 | x | x | 0.5% | 1000 | 147 | 57 | |
| | | | | | MC3 | | | 15 | | | | |
| 57 | 15 | 7 | x | x | 1 | x | 2 | 0.5% | 1000 | 163 | 41 | |
| | | | | | MC3 | | | 15 | | | | |
| 58 | 15 | 7 | x | x | 2 | x | 1 | 0.5% | 1000 | 162 | 41 | |
| | | | | | MC3 | | | 15 | | | | |
| 59 | 15 | 7 | x | 3 | 2 | x | x | 0.5% | 1000 | 149 | 45 | |
| | | | | | MC3 | | | 15 | | | | |
| 60 | 15 | 7 | x | 3 | 3 | x | x | 0.5% | 1000 | 177 | 43 | |
| | | | | | MC3 | | | 15 | | | | |
| 61 | 15 | 7 | x | 3 | 1 | x | x | 0.5% | 1000 | 144 | 45 | |
| | | | | | SM | | | 15 | | | | |
| 62 | 15 | 7 | x | 3 | 1 | x | x | 0.5% | 1000 | 139 | 44 | |
| | | | | | ALC | | | 15 | | | | |
| 63 | 15 | 7 | x | 1.5 | 2 | x | x | 0.5% | 1000 | 152 | 48 | |
| | | | | | MC3 | | | 15 | | | | |
| 64 | 15 | 7 | x | 3 | 1 | x | x | 0.5% | 1000 | 134 | 51 | |
| | | | | | MC3 | | | 15 | | | | |
| 65 | 15 | 7 | x | 1.5 | 2 | x | x | 0.5% | 1000 | 159 | 48 | |
| | | | | | MC3 | | | 15 | | | | |
| 66 | 15 | 7 | x | 1 | 3 | x | x | 0.5% | 1000 | 171 | 46 | |
| | | | | | MC3 | | | 15 | | | | |
| 67 | 15 | 7 | x | 0.5 | 3 | x | x | 0.5% | 1000 | 134 | 38 | |
| | | | | | MC3 | | | 15 | | | | |
| 68 | 15 | 7 | x | 1.5 | 2 | x | x | 0.5% | 1000 | 178 | 52 | |
| | | | | | MC3 | | | 15 | | | | |
| 69 | 15 | 7 | x | 1.5 | 2 | x | x | 0.5% | 1000 | 124 | 49 | |
| | | | | | ALC | | | 15 | | | | |
| 70 | 15 | 7 | x | 1.5 | 2 | x | x | 0.5% | 1000 | 125 | 53 | |
| | | | | | SM | | | 15 | | | | |
| 71 | 15 | 7 | x | 1.5 | 2 | x | x | 0.5% | 1000 | 146 | 53 | |
| | | | | | MC3 | | | 15 | | | | |

### Characterization of Nanoparticles

NP synthesis yield was determined by measuring the final weight of NP powders following lyophilization without sucrose. A dynamic light scattering (DLS) and zeta potential particle analyzer (Malvern Panalytical, Zetasizer Nano ZS) was used for the measurement of mean size, zeta potential, and polydispersity index (PDI) of the prepared nanoparticles. For DLS measurements, lyophilized NPs were redispersed in 200 μL of ultrapure water and then diluted 1:100 (v/v) in ultrapure water. For scanning electron microscopy (SEM, Thermo Scientific Quattro S) and transmission electron microscopy (TEM, JEOL 2100F) imaging, NPs were redispersed in 200 μL of ultrapure water and added directly to either silicon chips (Ted Pella, cat. 16008) for SEM imaging or carbon mesh (Ted Pella, cat. 01810) for TEM imaging. NPs were left to settle for at least one hour prior to imaging and excess NP solution was blotted with tissue paper. For SEM imaging, silicon chips with NPs were coated with a gold/palladium mixture using a sputter coater (Emitech K550). Note that it is expected that NPs will have a significant difference in hydrodynamic size and size measured through TEM as it is expected that the cationic surface will result in a significant hydration layer and therefore increased size when measured through DLS. For nanoparticle tracking analysis (NTA), an aliquot of lyophilized NPs was dispersed in 100 μL of ultrapure water and sent to the Brown University Extracellular Vesicle Core to be analyzed in a NanoSight NS500 (Malvern Panalytical).

mRNA Complexation and Gel Electrophoresis Assay

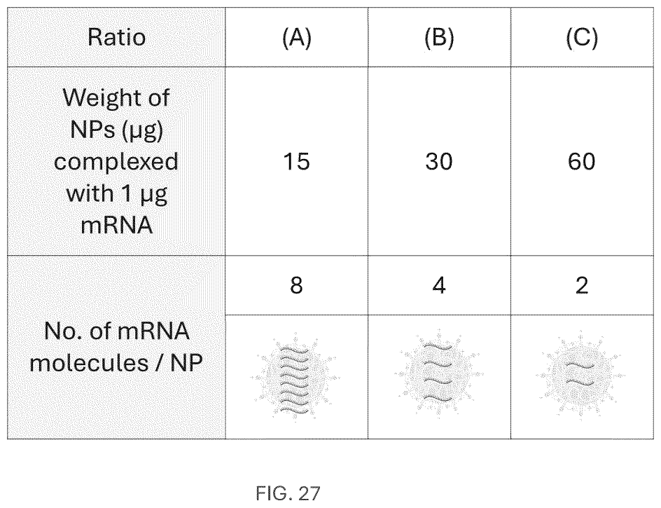

mRNA was mixed with NPs at specific ratios labeled as Ratio A: 15 μg of NPs to 1 μg of mRNA, Ratio B: 30 μg of NPs to 1 μg of mRNA, Ratio C: 60 μg of NPs to 1 μg of mRNA (FIG. 27). All complexation assays were performed with green fluorescent protein (GFP) mRNA (TriLink, cat. L-7201). mRNA stock solution (1 mg/mL in 1 mM sodium citrate buffer) was diluted 1:20 with RNAse/DNAse free water (Corning, cat. 46-000-CV) to a working concentration of 0.05 μg/μL. Lyophilized NPs were resuspended in appropriate volumes of RNAse/DNAse free water so that mixing at equal volumes with the working mRNA solution produces the desired ratios as previously described. For example, taking a lyophilized aliquot of NP-sucrose solution containing 300 μg of NPs and dispersing it in 200 μL of RNAse/DNAse free water results in a concentration of 1.5 μg/μL. Therefore, mixing NPs and mRNA at equal volumes at these concentrations would result in a solution corresponding to Ratio B. 1% agarose gels were prepared in 1× tris-buffered saline (Bio-Rad, cat. 1706435) with 5 μL of SYBR™ gold nucleic acid stain (Invitrogen, cat. S11494). 5 μL of BlueJuice™ gel loading buffer (Invitrogen, cat. 10816015) was mixed with 5 μL of mRNA alone or 10 μL of mRNA-NPs. Gel electrophoresis was performed using a gel box (Bio-Rad Mini-Sub Cell GT) and electrophoresis power supply (Bio-Rad, cat. 1645050) at 80 mA for 30 minutes. Gels were imaged via a Bio-Rad ChemiDoc imaging system using a blue screen sample tray (Bio-Rad, cat. 12003027). The band for mRNA complexed to NPs was expected to be retained in the well only due to the high molecular weight of the NPs, where any unbound mRNA in the mixture was expected to run down the gel at a similar distance to the control free mRNA.

Next, the effect of mixing time and mixing technique were qualitatively evaluated using gel electrophoresis to determine how quickly and simply the NPs can bind mRNA. For this experiment, NPs were mixed with GFP mRNA at Ratio A only. mRNA-NPs were either added to the gel after incubating for 5 minutes at room temperature or immediately after mixing. mRNA was mixed with NPs either through simple hand mixing (briefly shaking the tube containing the mRNA-NPs by hand), vortexing for 3 or 10 seconds, or not mixed at all (solutions pipetted together and added immediately to the gel). The strength of the mRNA complexation to NPs and the ability for NPs to protect mRNA from degradation by endogenous nucleases was also qualitatively measured via gel electrophoresis. mRNA-NPs were mixed at Ratio A. Nuclease reaction buffer (Tris-HCl pH 8.0, 5 mM CaCl2)) was supplemented to contain 1×BSA (Sigma-Aldrich, cat. A7030) as a stabilizing agent. Micrococcal nuclease (New England Biolabs, cat. M0247S) stock solution was diluted in reaction buffer to a 100× dilution and added to mRNA or mRNA-NPs to degrade 1 μg mRNA. This reaction solution was incubated in a shaker set to 100 rpm at room temperature for 15 minutes.

In the experimental group analyzing the ability of nucleases to break down NP-bound mRNA, nuclease activity was first stopped in the reaction solution via the addition of EGTA (RPI, cat. E14100-50.0) to a concentration of 20 mM. Next, heparin (Sigma-Aldrich, cat. H3393) was added to the reaction mixture to a final concentration of 25 mg/mL and incubated at room temperature for 15 minutes to release mRNA from the NPs. BlueJuice™ was added to the solution in a 1:10 (v/v) ratio and samples were run on a gel and imaged using the previously described protocol.

### Transfection

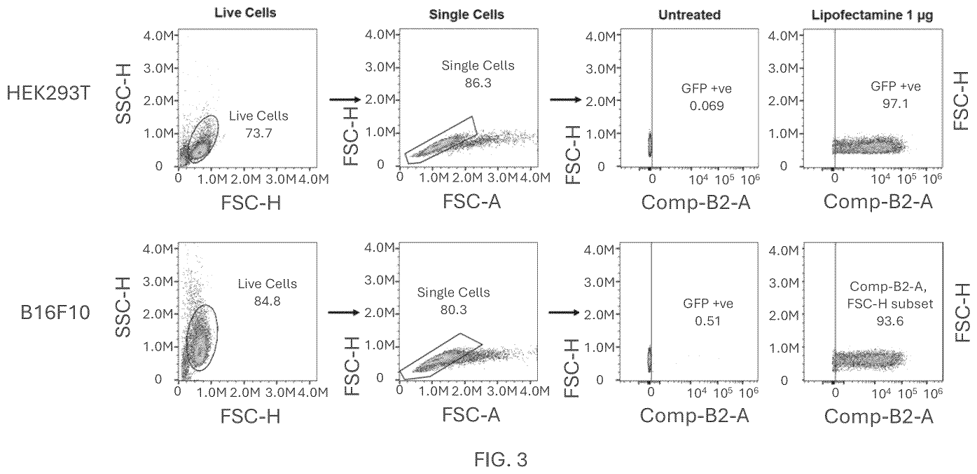

HEK293T cells were seeded on 24 well plates at 25,000 cells/well and allowed to grow for 24 hours. GFP mRNA was used as a model mRNA with easily detectable protein expression. GFP mRNA mixed with NPs was added directly to cells. After 48 hours, cells were collected for analysis via flow cytometry using the previously described method. GFP fluorescence was measured in the B2 channel. An example gating strategy is demonstrated in FIG. 3. Fluorescent microscopy images were taken on an ECHO Revolve 2 fluorescent microscope.

Transfection efficiency is the proportion of cells in a population that are transfected with mRNA and produce functional protein. Transfection magnitude is the degree of functional protein expression following transfection with genetic material.

Preparation of mRNA Lipid Nanoparticles (LNPs) for Cellular Transfection

1,2-distearoyl-sn-glycero-3-phosphocholine (DSPC) in chloroform was purchased from Avanti Research (cat. 890890C and 850365C). Chloroform was removed from this solution using rotary evaporation, leaving behind 100 mg of dried DSPC. 12.64 mL of ethanol (EtOH) was added to the DSPC powder to create a 10 mM solution. D-Lin-MC3-DMA ionizable lipid was made into a 39 mM solution in EtOH. Cholesterol was purchased from Sigma-Aldrich (cat. C3045) and dissolved in EtOH to a concentration of 20 mM. 1,2-dimyristoyl-rac-glycero-3-methoxypolyethylene glycol (DMG-PEG) with a molecular weight of 2 kDa was purchased from Avanti Research (cat. 880151P) and dissolved in EtOH to a concentration of 1 mM.

Complete lipid mixes (CLM) were prepared using a molar ratio of 50:10:39:1 of MC3/DSPC/Chol/PEG. To do this, we mixed 68.5 μL of MC3 stock, 53.3 μL of DSPC stock, 103.9 μL cholesterol stock, and 53.3 μL DMG-PEG stock. We then added 254 μl EtOH to this CLM. To make the MC3 LNPs, we added 21 μL of the CLM to an eppendorf tube and then added 9 μL EtOH. To another tube, we added 90 μL of 10 mM citrate buffer (10 mM, pH=4) and 10 μg of GFP mRNA from a 1 mg/mL stock solution. A vortex mixer was turned on at a moderate speed and the tube containing the citrate buffer and mRNA was placed on the mixer. The CLM mixture was pipetted quickly into the vortexing mRNA buffer solution and vortexed for 30 seconds. Following this, the resulting solution was incubated for 15 minutes at room temperature. The solution was dialyzed against 1×PBS in a beaker containing a stir bar on a stir plate set to 200 rpm using a Pur-A-Lyzer™ Midi Dialysis Kit with a molecular weight cutoff of 3.5 kDa (purchased from Sigma-Aldrich, cat. PURD35050) for one hour at room temperature. Afterwards, dialyzed LNP solutions were stored at 4° C. until use.

For transfection experiments, the LNP solution was diluted tenfold in 1×PBS. One-tenth the volume of this diluted LNP solution was added to wells containing 25,000 HEK293T cells for transfection. Assuming 100% encapsulation efficiency of the mRNA, this would correspond to each well receiving 1 μg of GFP mRNA, equivalent to what was delivered utilizing the NPs for comparisons.

### Statistical Analysis

All statistical analyses were performed using GraphPad Prism software v10.4.1. One way analysis of variance (ANOVA) was used to determine significance on all data unless otherwise indicated. Error bars in figures represent standard deviation unless otherwise indicated. P-values less than 0.05 were considered significant. Significance stars correspond to the following: *=p<0.05, **=p<0.005, ***=p<0.0005, ****=p<0.0001.

### Results

NPs made using this method were able to be synthesized with reproducible characteristics between batches. The mass of a single batch of the exemplary NP 71 formulation averaged around 9 mg, which was equal to approximately 50% yield. NP performance was further optimized via strict temperature control during handling of the MC3 lipids and the shielding of lipids from light throughout the process. NTA revealed that the number of NPs produced in a single batch was approximately 1.56×10¹⁴ particles and that when complexing GFP mRNA to NPs at Ratio A the ratio of mRNA molecules to NPs was about 7:1.

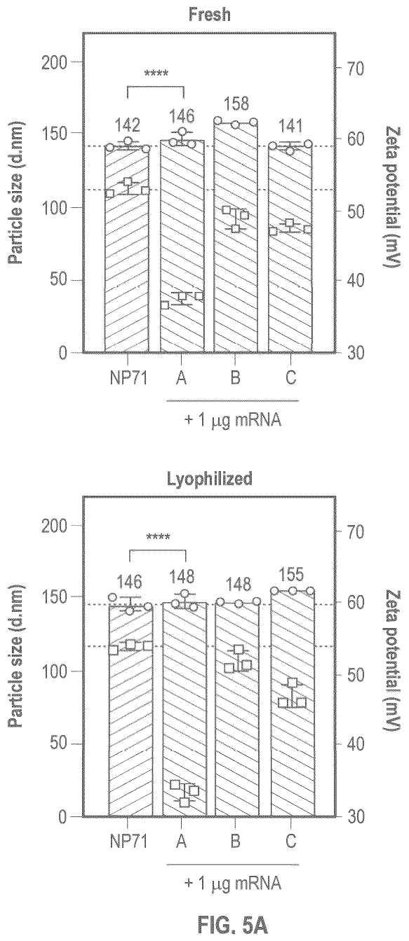

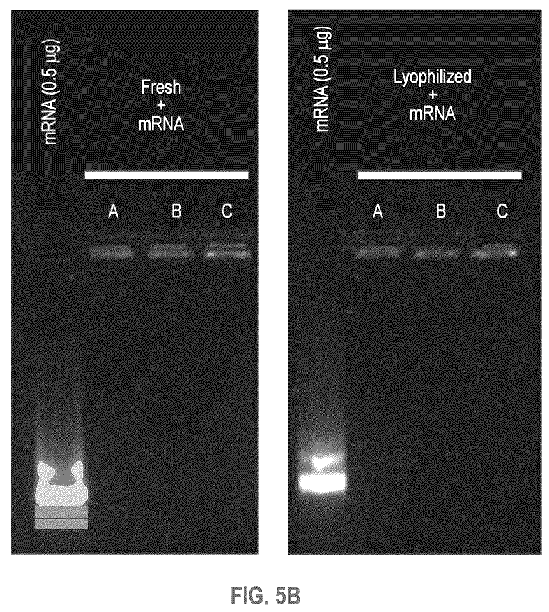

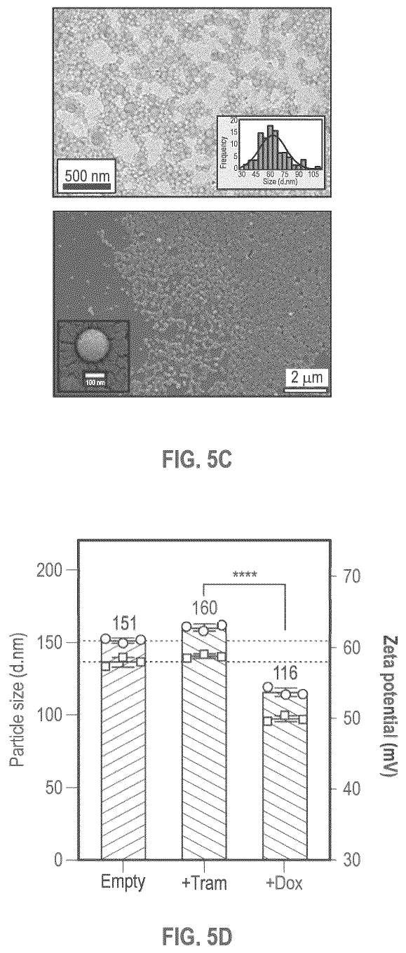

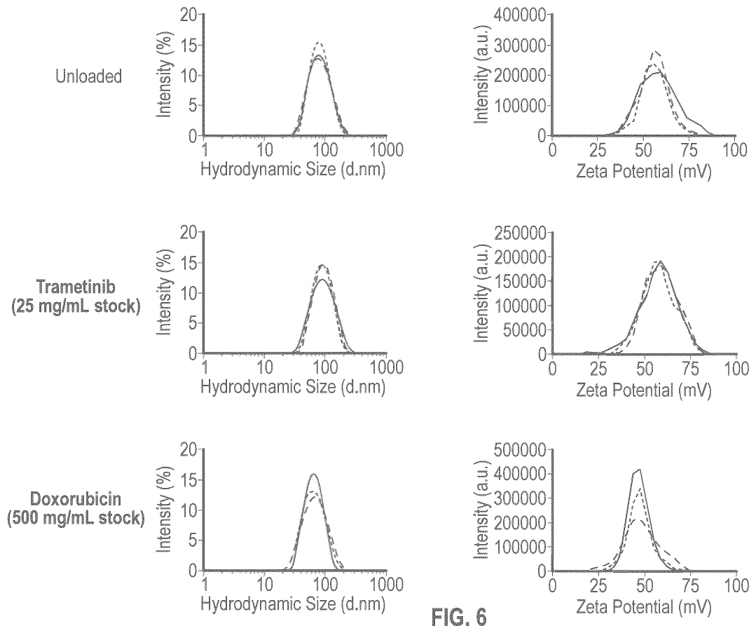

The average hydrodynamic size of non-drug-loaded NP 71 following lyophilization and reconstitution was 145.9±4.6 nm, average PDI was 0.167±0.018, and average zeta potential was 53.0±0.8 mV (FIG. 5A). Frequency distribution curves for this data can be found in FIG. 6. These values were consistent with the DLS measurements taken of NPs pre-lyophilization, indicating that the lyophilization process did not significantly alter the major physical properties of these NPs.

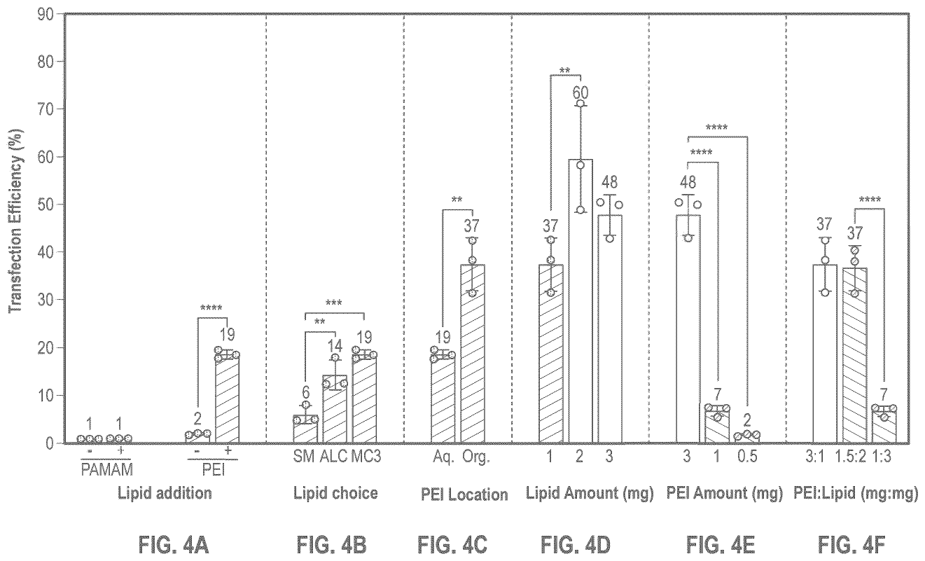

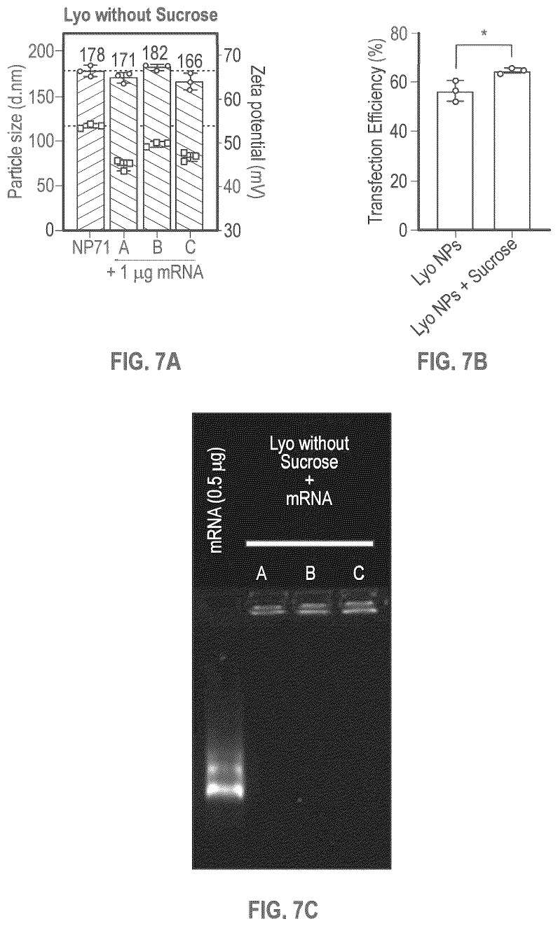

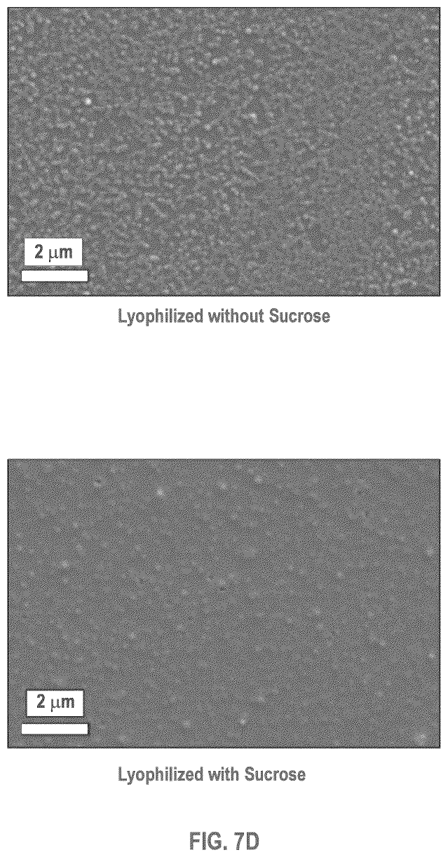

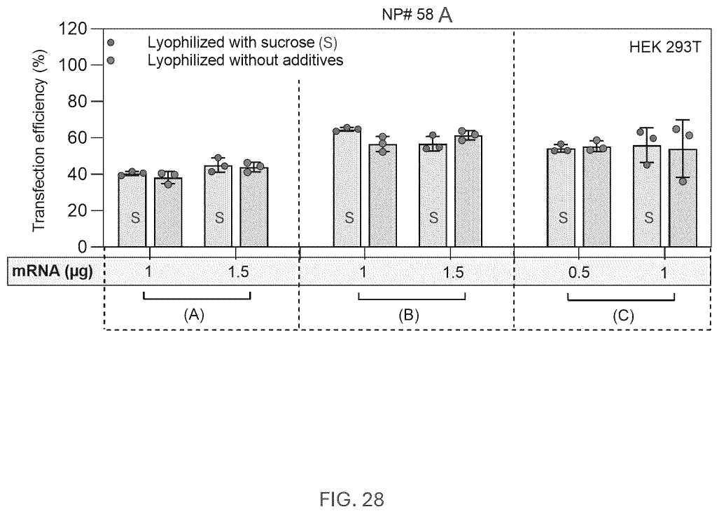

Complexation of NPs with GFP mRNA through simple hand mixing did not alter their hydrodynamic size and NPs remained positively charged at all ratios (FIG. 4A). Gel electrophoresis demonstrated that NP 71 could completely complex mRNA at all ratios and that lyophilization did not affect the binding activity of these NPs (FIG. 5B). TEM imaging of NP 71 was used to determine NP size distribution data, with an average size of 62.5±14.3 nm (FIG. 5C, top). SEM imaging further confirmed that the synthesized NPs were morphologically spherical and of relatively uniform size and shape (FIG. 5C, bottom). Characterization data for NPs lyophilized without sucrose are provided in FIGS. 7A-7C. FIG. 28 shows that NPs are able to transfect cells after lyophilization, and that the transfection efficiency of NPs lyophilized with and without sucrose are similar.

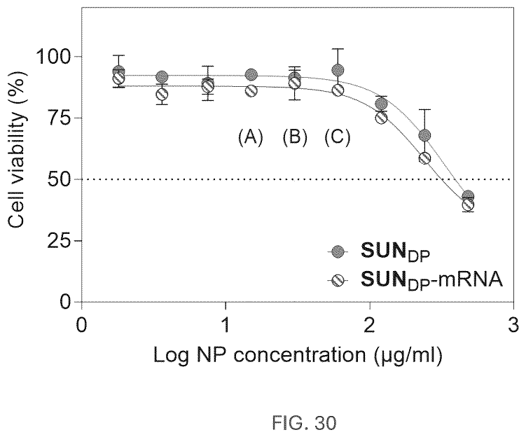

FIGS. 4A-4F show the transfection efficiency of exemplary formulations 4, 5, 36, 55, 53, 54, 55, 55, 56, 56, 59, 60, 60, 66, 67, 56, 63, and 66 (from left to right). In FIG. 4A, PLGA+PAMAM and PLGA+PEI formulations were tested with or without MC3 (lipid). An increase in transfection was achieved when PEI was used in the presence of the ionizable lipid. In FIG. 4B, PLGA+PEI formulations were tested with SM, ALC, or MC3 lipids. MC3 had the highest transfection efficiency. In FIG. 4C, the PLGA+PEI+MC3 nanoparticles were made with PEI added either during the aqueous or organic phase. PEI addition in the organic phase resulted in higher transfection efficiency. In FIG. 4D, different amounts of MC3 were tested. Higher amounts of MC3 resulted in higher transfection efficiency, as well as low cell viability (indicated by red bars). In FIG. 4E, the amount of PEI was changed. Higher amounts of PEI resulted in higher transfection efficiency, but low cell viability (indicated by red bars). In FIG. 4F, ratios of PEI:lipid at 3:1, 1.5:2, and 1:3 were tested. The ratio of 1.5:2 had the highest transfection efficiency with high cell viability (indicated by green bars). FIG. 30 shows that cell viability is similar when cells are exposed to NPs with and without mRNA.

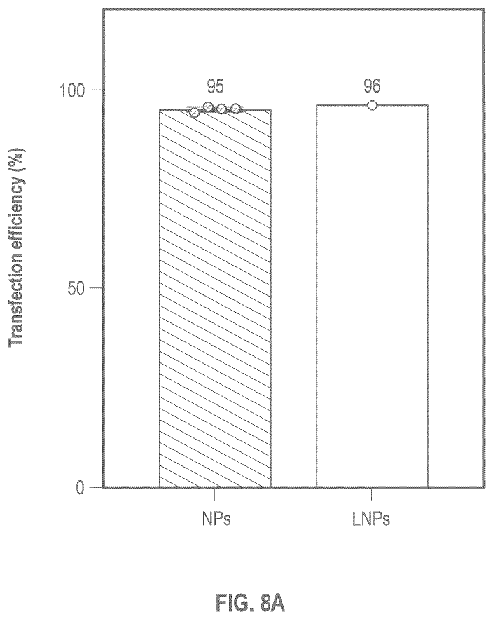

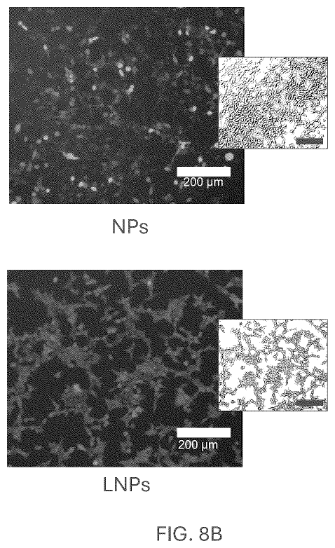

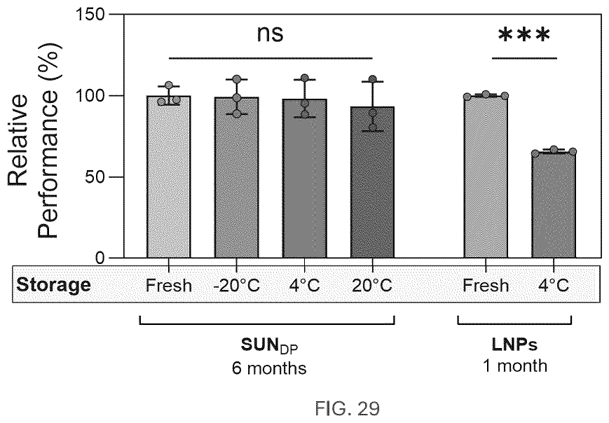

Moreover, when compared to lipid nanoparticles, NP 71 demonstrated a comparable transfection efficiency (FIGS. 8A and 8B). NPs prepared with GFP mRNA at ratios A and B demonstrated comparable transfection efficiency to LNPs, with experiments repeated in triplicate. To compare the stability of NPs versus LNPs, lyophilized NP powders were stored for 6 months at either −20° C., 4° C., or room temperature (20° C.) and LNPs were stored in buffer at 4° C. for 1 month. Following this, HEK293T cells were incubated with reconstituted NP complexed to 1 μg of GFP-mRNA at Ratio A or a dose of stored LNPs delivering a total of 1 μg of encapsulated GFP-mRNA for 48 h. Transfection efficiencies were measured using flow cytometry and results were normalized to the transfection efficiencies of the freshly prepared NP (SUN_{DP}) and LNP formulations. Statistical analysis was carried out using repeated-measures one-way ANOVA with the Gessier-Greenhouse correction with a Tukey post hoc test for comparisons between SUN_{DP} groups and paired two-sided t-test for comparisons between LNP groups. FIG. 29 shows that NPs stored at −20 C, 4 C, and 20 C for 6 months maintained transfection efficiency, while LNPs stored for only 1 month at 4 C showed a decreased transfection efficiency.

Altogether, these results demonstrate the production of exemplary nanoparticle formulations, and their ability to transfect cells with mRNA.

### Example 2. Nanoparticle Complexation with mRNA

This Example demonstrates that nanoparticles rapidly complex with mRNA and complexation protects mRNA from degradation by an exemplary nuclease. Nanoparticles were produced as described in Example 1.

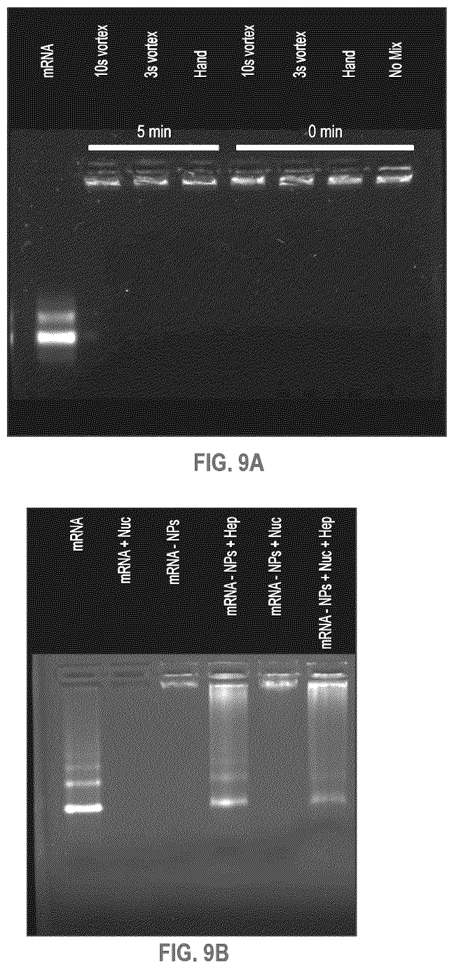

NPs were mixed with mRNA either by hand or vortexing for 3 or 10 seconds, incubated for 5 minutes or had no incubation time, and were added to gels to observe the completeness of their complexation. One group of NPs was not mixed at all with the mRNA, instead mRNA was pipetted to the NP solution and loaded immediately onto the gel. All groups demonstrated complete binding of mRNA, indicating that these NPs were able to rapidly bind to mRNA in solution without the need for long mixing or incubation times (FIG. 9A).

Next, the ability for NPs to protect mRNA from digestion by nucleases was observed (FIG. 9B). Wells are numbered 1-6 left to right. Well 1 contained free mRNA only. In well 2 the nucleases were able to break down the mRNA, causing it to run down and off the gel. Well 3 demonstrates the ability for NPs to bind to mRNA and well 4 demonstrates that heparin can be used to release mRNA from the surface of the NPs. In well 5, despite the presence of nucleases, mRNA did not run down the well and off the gel when bound to NPs. Well 6 shows, after the mRNA was released by heparin, the same bands as seen in well 4 are observed, demonstrating that the mRNA bound to the NP was not degraded by the nuclease. These results indicate that the binding of mRNA to these NPs would likely offer protection from degradation from endogenous nucleases in the body, meaning that mRNA may be able to survive intact in the body after injection and be able to be taken into cells to produce functional proteins.

As shown in FIG. 9B, heparin only slightly detaches mRNA from the surface of the NPs despite its extremely high negative charge. Without wishing to be bound by theory, these data suggest that the mRNA was very tightly complexed with the NPs, and that it is not expected that any anionic endogenous factors in the body would prematurely release the mRNA from the NPs through competitive electrostatic binding since these factors would carry a lesser charge.

### Example 3: Nanoparticle Loading with Trametinib and Doxorubicin

This Example describes the loading of two exemplary small molecules, trametinib and doxorubicin.

Drug stock solutions in DMSO were prepared at their solubility limits following ultrasonication and gentle warming: 25 mg/mL for trametinib and 500 mg/mL for doxorubicin. Nanoparticles were prepared as described in Example 1, with 10 μL of the drug stock solutions added to the organic phase prior to the NP dripping stage in nanoprecipitation.

### Characterization of Nanoparticles

Encapsulation efficiencies of drug-loaded NPs were measured utilizing high-performance liquid chromatography (HPLC) with UV-vis detection (Agilent 1260 Infinity II). Our HPLC method was modified from Saklani et al., 2022 [111]. Briefly, we created a 0.025% (w/v) octane-sulfonic acid (Thermo Scientific, cat. 206140050) buffer solution adjusted to a pH of 2.6 or below using phosphoric acid (EMD Millipore, cat. 100573). HPLC-grade acetonitrile (VWR, cat. BDH83639.400) was used as the other component of the mobile phase. The final composition of the mobile phase was 60:40 (v/v) buffer-acetonitrile. A C18 reversed-phase column (Sepax Bio-C18, cat. 106185-4625; 4.6×250 mm, pore size 5 μm) held at 40° C. was used for all runs. The flow rate was set to 1 mL/min and sample injection volume was 20 μL. Diluent for free drugs or drug-loaded NPs was 50:50 (v/v) water-acetonitrile. Doxorubicin detection absorbance was set at 234 nm and trametinib detection absorbance was set at 250 nm.

### Results

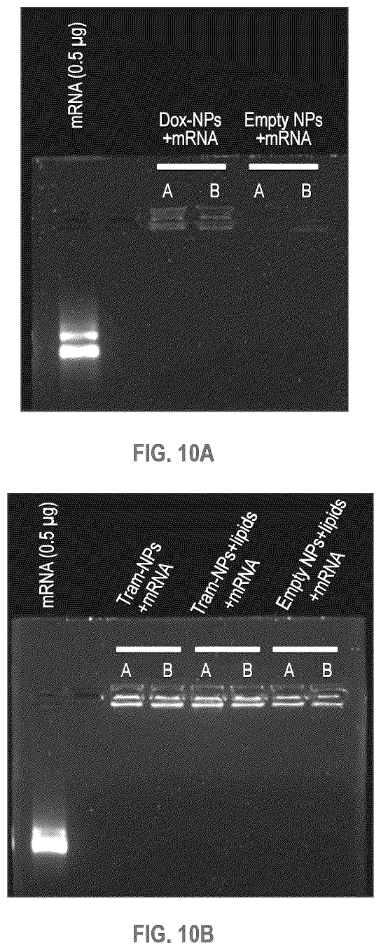

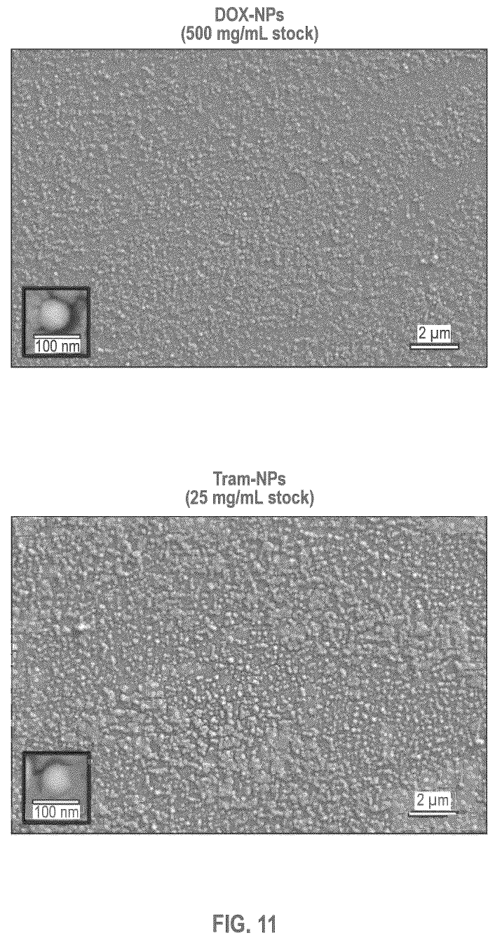

Trametinib-loaded NPs (Tram-NPs) made with the 25 mg/mL stock solution had consistent hydrodynamic size and shape to that of the unloaded NPs and a PDI of 0.170±0.006. The doxorubicin NPs made with the 500 mg/mL stock solution had reduced size and charge with a PDI of 0.150±0.023 (FIG. 5D). Representative intensity distribution curves for this data can be found in FIG. 6. Encapsulation of drugs did not impact the ability for these NPs to complex mRNA (FIGS. 10A and 10B). SEM images for trametinib- and doxorubicin-loaded NPs are shown in FIG. 11.

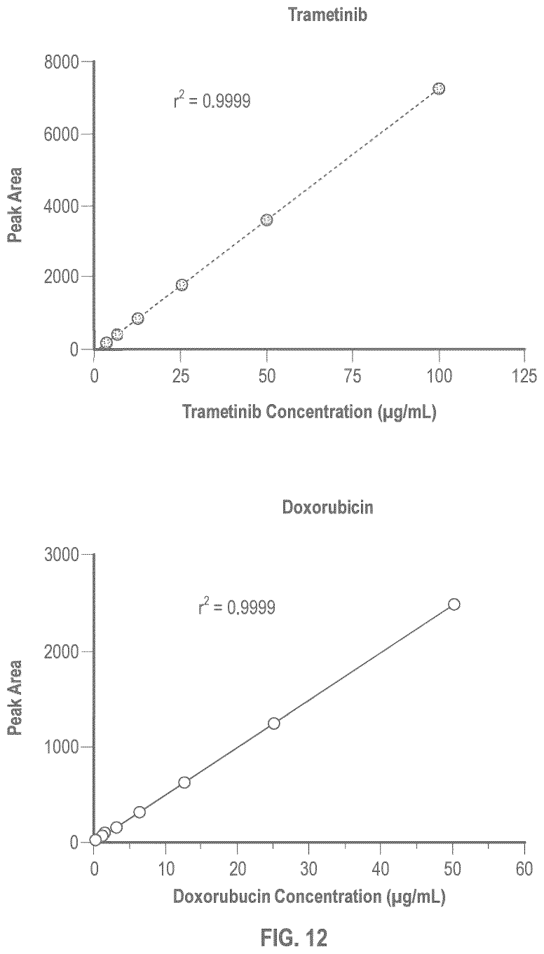

Both trametinib and doxorubicin free drugs had highly linear calibration curves in HPLC UV-vis detection using the previously described method with coefficients of correlation greater than 0.99 (FIG. 12). Drug-loaded NPs in diluent were run in HPLC and absorbances correlated to drug concentrations using these calibration curves. A summary of nanoparticle drug encapsulation efficiencies and drug loadings is provided in Table E2.

TABLE E2
Drug Encapsulation and Loading into NPs at Variable Drug Stock
Solution Concentrations Used During NP Synthesis. Reported
data are means plus and minus the standard deviation. Encapsulation
efficiency and drug loading percent is (w/w)

| | Stock | Total Weight | Encapsula- | |
|---|---|---|---|---|
| | Concentra- | of Drug | tion | Drug |
| | tion | Encapsulated | Efficiency | Loading |
| Drug | (mg/mL) | (ug) | (%) | (%) |
| Doxorubicin | 250 | 33.0 ± 5.73 | 1.32 ± 0.23 | 0.37 ± 0.064 |
| | 500 | 71.0 ± 2.43 | 1.42 ± 0.049 | 0.79 ± 0.027 |
| Trametinib | 25 | 102.5 ± 27.2 | 41.0 ± 10.9 | 1.14 ± 0.30 |
| | 50 | 198.9 ± 18.4 | 39.8 ± 3.68 | 2.21 ± 0.20 |

Doxorubicin-loaded NPs (Dox-NPs) made with the 500 mg/mL stock had an average of 71.0±2.4 μg of encapsulated drug, corresponding to an encapsulation efficiency of 1.42±0.049%. This encapsulation efficiency can be attributed to the relatively high water-solubility of doxorubicin, meaning that the majority of the drug in the organic phase will distribute into the aqueous phase and not be trapped inside of the NPs upon precipitation of the polymers. Tram-NPs made with the 25 mg/mL stock had an average of 102.5±27.2 μg of encapsulated drug, corresponding to an encapsulation efficiency of 41.0±10.9%.

Overall, NPs with acceptable morphology and surface containing either doxorubicin or trametinib were synthesized, confirming the capability of these NPs to be loaded with therapeutic drugs.

### Example 4: Delivery of Trametinib to Cells In Vitro with Nanoparticles

This Example describes the delivery of two exemplary small molecules, trametinib and doxorubicin, to cells in vitro.

### Transfection

B16F10 cells were seeded on 24 well plates at 25,000 cells/well and allowed to grow for 24 hours. GFP mRNA was used as a model mRNA with easily detectable protein expression. GFP mRNA mixed with NPs was added directly to cells. After 48 hours, cells were collected for analysis via flow cytometry using the previously described method. GFP fluorescence was measured in the B2 channel. Fluorescent microscopy images were taken on an ECHO Revolve 2 fluorescent microscope.

### Results

### Effect Offree Trametinib and Doxorubicin In Vitro

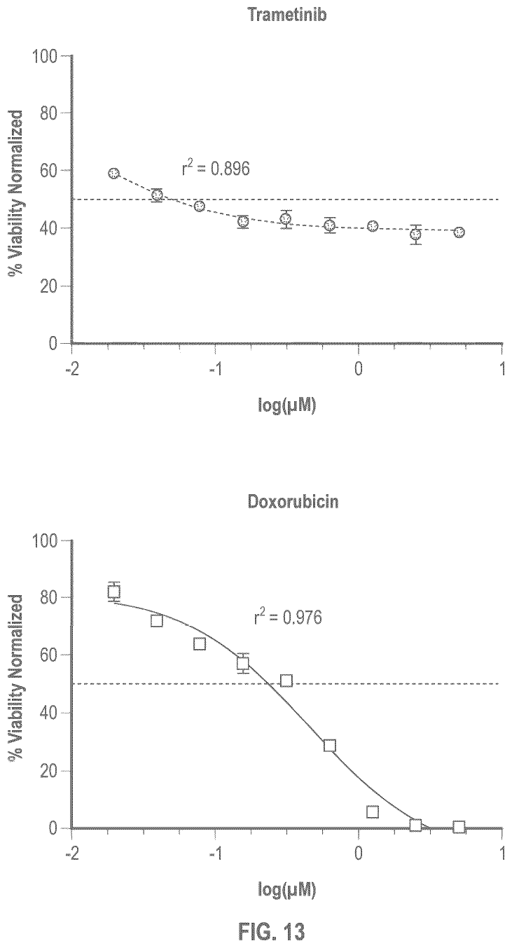

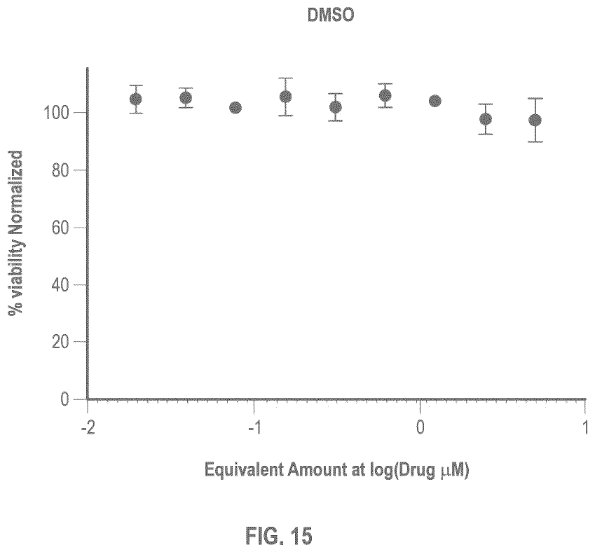

First, drug dose-response curves using the drug dissolved in DMSO were generated after 72 hours of incubation of cells with the drugs and fit to a three-parameter nonlinear regression using the built-in regression model in GraphPad Prism (FIG. 13). The half-maximal inhibitory concentration, or IC50, of trametinib was 0.05 μM while the IC50 of doxorubicin was 0.45 μM. The dose-response curve of DMSO alone is provided in FIG. 15, where it can be observed that the presence of DMSO had no significant impact on the viability of these cells. While trametinib had a significantly lower IC50 than doxorubicin, the dose-response curve indicates that increasing the trametinib concentration beyond a certain point did not further decrease the cell viability, plateauing at around 40% cell viability. As mentioned previously non-BRAF mutated melanoma cells like B16F10 have been reported to be at least partially responsive to trametinib treatment, however reports are conflicting. This could explain the conflicting information because while these cells were sensitive to trametinib, especially at lower doses, at 40% viability these cancer cells may be able to recover from treatment meaning total elimination of non-BRAF mutated cells with trametinib may not be possible. This indicates the potential for a dual-therapy approach, as described herein, with trametinib that could eliminate the remaining tumor cells, thus enabling trametinib therapy for a broader range of tumors.

### Tram-NPs Kill Melanoma Cells

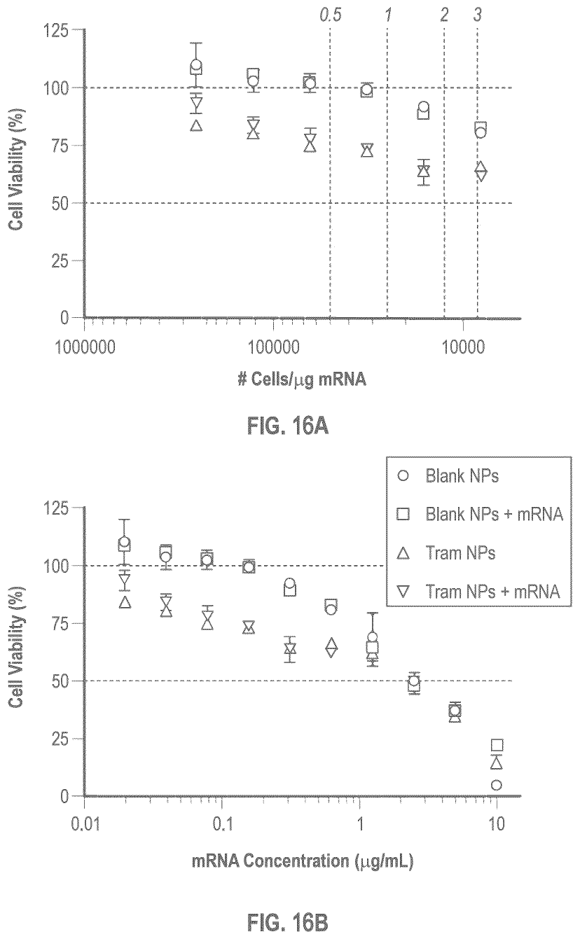

NPs were either complexed with mRNA at Ratio B as described in Example 1 (FIG. 27) or diluted with an equivalent amount of water and were incubated with B16F10 cells for 48 hours before viability was measured. Dose-response curves for non-drug-loaded NPs (Blank NPs) and Tram-NPs with and without mRNA are shown in FIGS. 16A and 16B. The numbers above the dashed lines in FIG. 16A correspond to equivalent g mRNA doses at Ratio B if this experiment were to have been carried out in 24-well plates containing 25,000 cells/well (for example, 12,500 cells/μg mRNA is equal to adding 2 μg of mRNA and 60 μg of NPs onto one of the wells of the 24-well plate), allowing us to correlate the viability results from this 96-well plate experiment to what would be expected for 24-well plates used for cell transfection experiments. It can be seen that non-drug-loaded NPs remain acceptably safe to these cells at Ratio B delivering mRNA doses up to 8000 cells per μg of mRNA, with cell viabilities around 810% for NPs loaded with mRNA and 80% for NPs without mRNA. As expected, Tram-NPs demonstrated decreased viability as trametinib is toxic to B16F10, where at Ratio B and 8000 cells per g of mRNA the Tram-NPs loaded with mRNA had cell viability around 62% whereas Tram-NPs without mRNA had cell viability around 66%. Importantly, this data demonstrates no significant difference between the toxicity of NP treatments that are or are not complexed with mRNA, indicating that losses in cell viabilities can be attributed to the toxic effects of the NPs themselves.

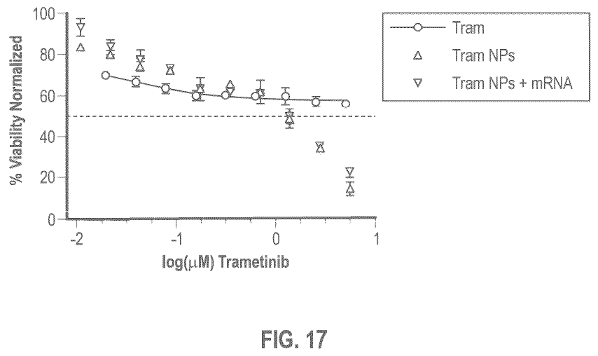

FIG. 16B shows that the IC50 was around 2.5 μg/mL of mRNA which was mixed with 75 μg/mL of NPs. FIG. 17 shows cell viability against the molar concentration of drug loaded into the Tram-NPs compared against free trametinib of equal concentrations. This data reveals that while soluble trametinib fails to bring B16F10 cell viability below 50% after incubation for 48 hours, Tram-NPs were able to achieve greater toxicity against these cells at higher doses, with an IC50 of 1.39 μM trametinib. In addition, at low NP doses, Tram-NPs had reduced inhibitory effect against B16F10 compared to the free drug. This could be attributed to release kinetics of the drug from the NPs, in which the drug is released over some period of time to the cells.

### Example 5: Nanoparticle Uptake In Vitro

This Example describes the uptake of NPs in vitro over time at different concentrations. Nanoparticles were made and complexed with mRNA as described in Example 1.

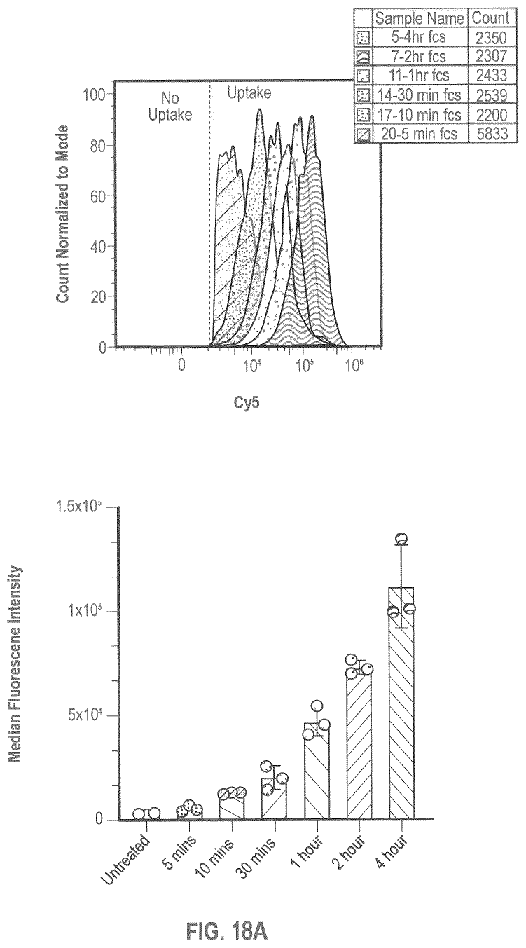

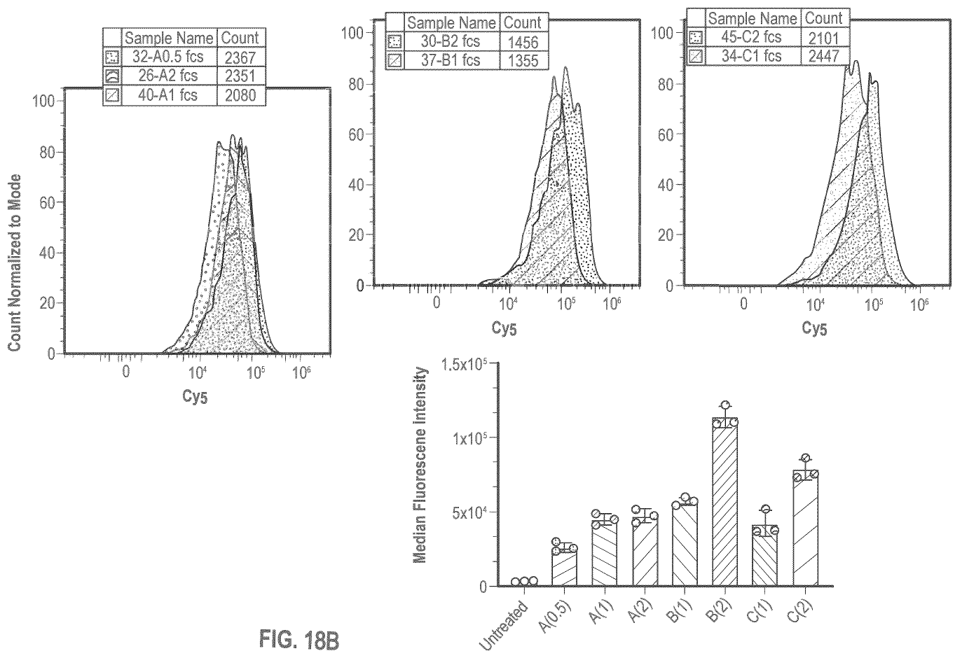

NPs without encapsulated drugs mixed with mRNA at Ratio A (FIG. 27) demonstrated time-dependent uptake in HEK293T cells (FIG. 18A). At 5 minutes the average percentage of cells that had taken up NPs was 61.8±20.8%. After 10 minutes, nearly all of the cells had taken up particles (at least 97% uptake for all wells at every time point beyond 10 minutes). In addition, the magnitude of cellular uptake of NPs increased as incubation time increased, indicating that incubation time is a factor in determining the magnitude of NP uptake into cells and therefore expected magnitude of protein expression.

mRNA-NPs at higher mRNA doses increases (and thus higher NP doses) increases NP uptake, as well (FIG. 18B). This was true across all mRNA to NP ratios, indicating that dose of NPs is a factor in cellular uptake. Notably, higher ratios of NPs to mRNA exhibited higher uptake except for Ratio C, which was likely due to increased cellular toxicity at such high NP amounts. Overall, this indicates that using more NPs to deliver the same amount of mRNA could lead to faster or higher degrees of cellular uptake up to a certain threshold.

### Example 6. NPs are Capable of Escaping Endosomes

This Example demonstrates that NPs were able to escape the endosomes of HEK293T and B16F10 cells. NPs were made as described in Example 1, complexed with Cy5-mRNA, and administered to HEK293T and B16F10 cells, as described below.

### Cellular Uptake and Endosomal Escape Assays

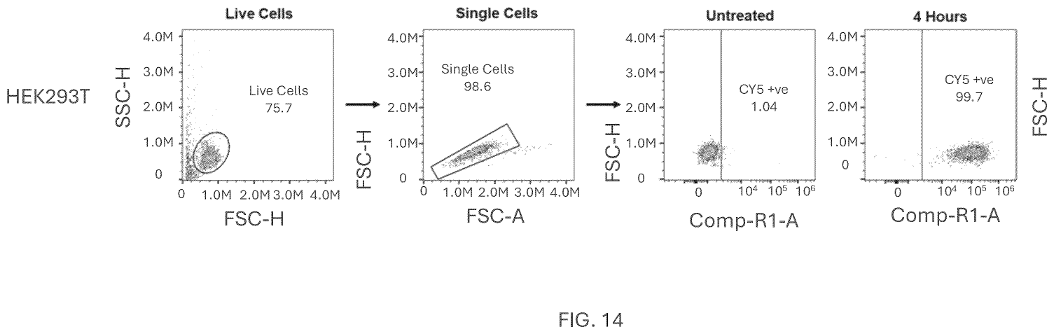

To analyze cellular uptake, Cy5 luciferase mRNA (ApexBio, cat. R1010) was used to track the location of mRNA following complexation to NPs. HEK293T cells were seeded onto 24 well cell culture plates (Genesee Scientific, cat. 25-107) at 25,000 cells/well and incubated for 24 hours. For the experiment analyzing the effect of incubation time on cell uptake, all mRNA-NPs were prepared at Ratio A (FIG. 27) and all wells received 1 μg total of mRNA. For the experiment analyzing the effect of mRNA-NP concentration on cell uptake, mRNA-NPs were prepared at the indicated ratios and incubated with cells for 1 hour. Here, Ratio D indicates a ratio of 7.5 μg of NPs to 1 μg of mRNA. mRNA-NPs were added to cells at specific time points so that all incubation periods ended at the same time and cells were collected. The cell collection protocol was as follows: Each well was washed with 250 μL of 1×PBS three times, after which 100 μL of trypsin-EDTA was added to each well. Plates were incubated for 3 minutes at 37° C., after which 400 μL of DMEM media was added to quench the enzyme. A pipette was used to vigorously agitate the cell solution, after which all cell solution was collected and added to flow cytometry tubes (Alkali Scientific, cat. CT6414). Tubes were kept on ice and briefly vortexed before being analyzed in a Cytek Aurora 4 laser (16UV-16V-14B-8R) flow cytometer. Cy5 signal was measured in channel RI. An example gating strategy is provided in FIG. 14. Data was analyzed using FlowJo software v10.10 (BD Biosciences).

For endosomal escape assays, HEK293T or B16F10 cells were seeded in a 4 well cell culture chambered microscopy slide (Ibidi, cat. 80426) at 100,000 cells/well. Following incubation for 24 hours at 37° C., old cell media was aspirated and cells were washed once with 100 μL 1×PBS. PBS was aspirated and replaced with fresh media. mRNA-NPs at the indicated ratios were added to cells for at least 1 hour. LysoTracker™ green stain (Thermo Scientific, cat. L7526) was added to cell media according to manufacturer instructions and incubated for 1 hour. Following this, cells were washed twice with 100 μL 1×PBS, PBS was aspirated, and fresh media was added to cells. Hoechst (Invitrogen, cat. H1399) nuclear stain was added to cell media according to manufacturer instructions. Cells were then taken immediately for imaging using a confocal microscope (Zeiss LSM800).

### Results

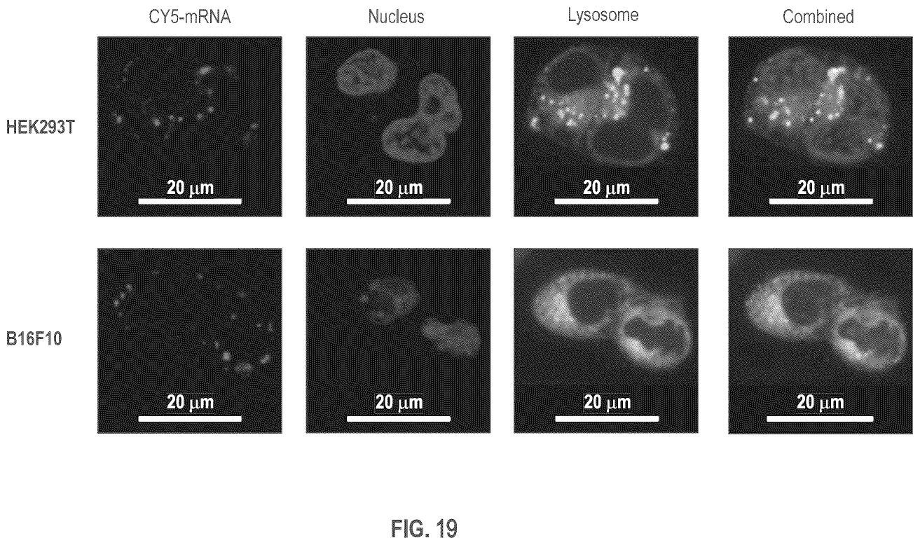

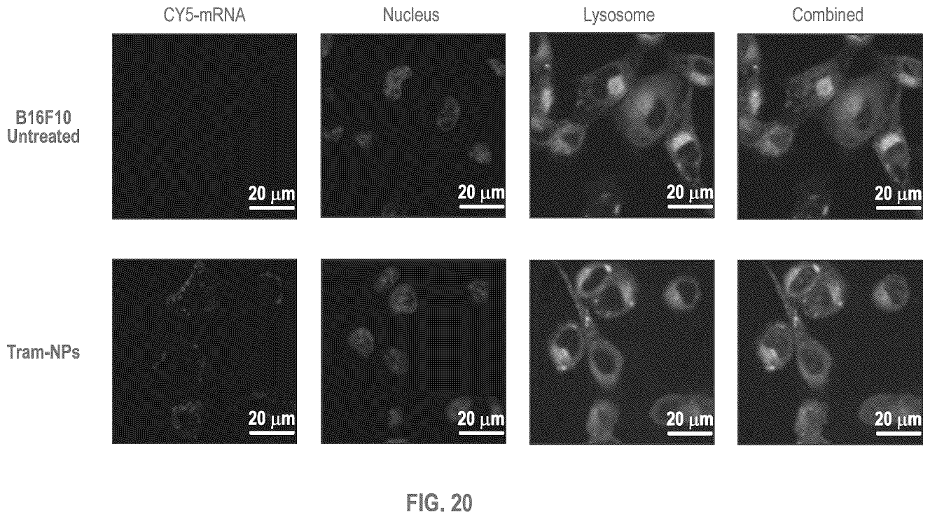

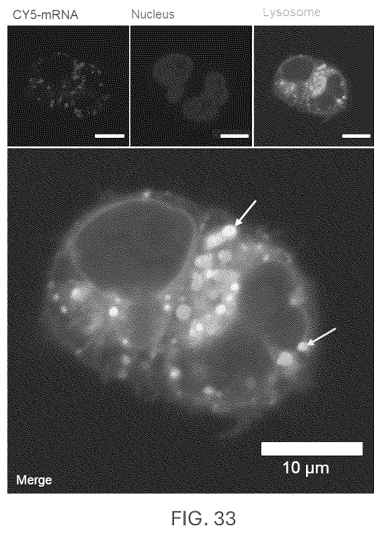

In treated cells, Cy5-mRNA signal was observed in the cytoplasm (FIGS. 19 and 33). In untreated cells no Cy5-mRNA signal was observed, indicating that this signal was specific to Cy5-mRNA-NPs (FIG. 20). It was also observed that Tram-NPs complexed to Cy5-mRNA were also able to escape cell endosomes, meaning drug loading did not hinder this activity (FIG. 20).

### Example 7. Transfection of B16F10 with Trametinib or Doxorubicin and GM-CSF mRNA-NPs

This Example demonstrates that nanoparticles can deliver trametinib and GM-CSF mRNA, or doxorubicin and GM-CSF mRNA, to cells in vitro. Nanoparticles were prepared as described in Example 1 and trametinib or doxorubicin were loaded as described in Example 3.

GM-CSF mRNA Synthesis

GM-CSF mRNA transcripts were obtained using ApexBio's custom mRNA synthesis service. Mus Musculus GM-CSF mRNA sequence was obtained from GenBank (accession number: EU366957, SEQ ID NO: 1).

TABLE E3
EU366957.1 Mus musculus granulocyte-macrophage
colony stimulating factor 2 (Csf2)
mRNA, complete cds

ATGTGGCTGCAGAATTTACTTTTCCTGGGCATTGTGGTCTACAGC
CTCTCAGCACCCACCCGCTCACCCATCACTGTCACCCGGCCTTGG
AAGCATGTAGAGGCCATCAAAGAAGCCCTGAACCTCCTGGATGAC
ATGCCTGTCACGTTGAATGAAGAGGTAGAAGTCGTCTCTAACGAG
TTCTCCTTCAAGAAGCTAACATGTGTGCAGACCCGCCTGAAGATA
TTCGAGCAGGGTCTACGGGGCAATTTCACCAAACTCAAGGGCGCC
TTGAACATGACAGCCAGCTACTACCAGACATACTGCCCCCCAACT
CCGGAAACGGACTGTGAAACACAAGTTACCACCTATGCGGATTTC
ATAGACAGCCTTAAAACCTTTCTGACTGATATCCCCTTTGAATGC
AAAAAACCAGGCCAAAAATGA
(SEQ ID NO: 1)

GM-CSF mRNA was generated with ApexBio's proprietary optimized untranslated regions, a Cap-1 (m7GpppNm-) 5′ cap, a 100-nucleotide poly(A) tail, and 100% N1-methylpseudouridine substitution. The provided QC report indicated that the mRNA we received was of acceptable purity (validated through gel electrophoresis), had a concentration of about 1.15 mg/mL in 1 mM sodium citrate buffer (pH of 6.4), and an A260/A280 of 1.96. A sample of the mRNA was analyzed upon receipt in a microvolume UV-vis spectrophotometer (Thermo Scientific, NanoDrop One) and the reported mRNA concentration and A260/A280 values were confirmed.

mRNA Complexation

mRNA was mixed with NPs at specific ratios labeled as Ratio A: 15 μg of NPs to 1 μg of mRNA, Ratio B: 30 μg of NPs to 1 μg of mRNA, Ratio C: 60 μg of NPs to 1 μg of mRNA (FIG. 27) (either green fluorescent protein (GFP) mRNA (TriLink, cat. L-7201) or GM-CSF mRNA). mRNA stock solution (1 mg/mL in 1 mM sodium citrate buffer) was diluted 1:20 with RNAse/DNAse free water (Corning, cat. 46-000-CV) to a working concentration of 0.05 μg/μL. Lyophilized NPs were resuspended in appropriate volumes of RNAse/DNAse free water so that mixing at equal volumes with the working mRNA solution produces the desired ratios as previously described. For example, taking a lyophilized aliquot of NP-sucrose solution containing 300 μg of NPs and dispersing it in 200 μL of RNAse/DNAse free water results in a concentration of 1.5 μg/μL. Therefore, mixing NPs and mRNA at equal volumes at these concentrations would result in a solution corresponding to Ratio B. 1% agarose gels were prepared in 1× tris-buffered saline (Bio-Rad, cat. 1706435) with 5 μL of SYBR™ gold nucleic acid stain (Invitrogen, cat. S11494). 5 μL of BlueJuice™ gel loading buffer (Invitrogen, cat. 10816015) was mixed with 5 μL of mRNA alone or 10 μL of mRNA-NPs. Gel electrophoresis was performed using a gel box (Bio-Rad Mini-Sub Cell GT) and electrophoresis power supply (Bio-Rad, cat. 1645050) at 80 mA for 30 minutes. Gels were imaged via a Bio-Rad ChemiDoc imaging system using a blue screen sample tray (Bio-Rad, cat. 12003027). The band for mRNA complexed to NPs was expected to be retained in the well only due to the high molecular weight of the NPs, where any unbound mRNA in the mixture was expected to run down the gel at a similar distance to the control free mRNA.

### Cellular Transfection Assays

HEK293T and B16F10 cells were seeded on 24 well plates at 25,000 cells/well and allowed to grow for 24 hours. GFP mRNA was used as a model mRNA with easily detectable protein expression. GFP mRNA was mixed with NPs at the indicated ratios and added directly to cells at the indicated mRNA doses. Lipofectamine (Thermo Scientific, cat. LMRNA003) was used as a positive control for transfection and used according to manufacturer instructions. After 48 hours, cells were collected for analysis via flow cytometry using the previously described method. GFP fluorescence was measured in the B2 channel. Fluorescent microscopy images were taken on an ECHO Revolve 2 fluorescent microscope.

The same protocol was followed for transfecting B16F10 cells with GM-CSF mRNA. After 48 hours, cell media from each well was collected for measurement of GM-CSF protein concentration using ELISA. A commercial murine GM-CSF ABTS ELISA kit (PeproTech, cat. 900-K55K) was purchased and used according to manufacturer instructions.

Transfection efficiency is the proportion of cells in a population that are transfected with mRNA and produce functional protein. Transfection magnitude is the degree of functional protein expression following transfection with genetic material.

### Results

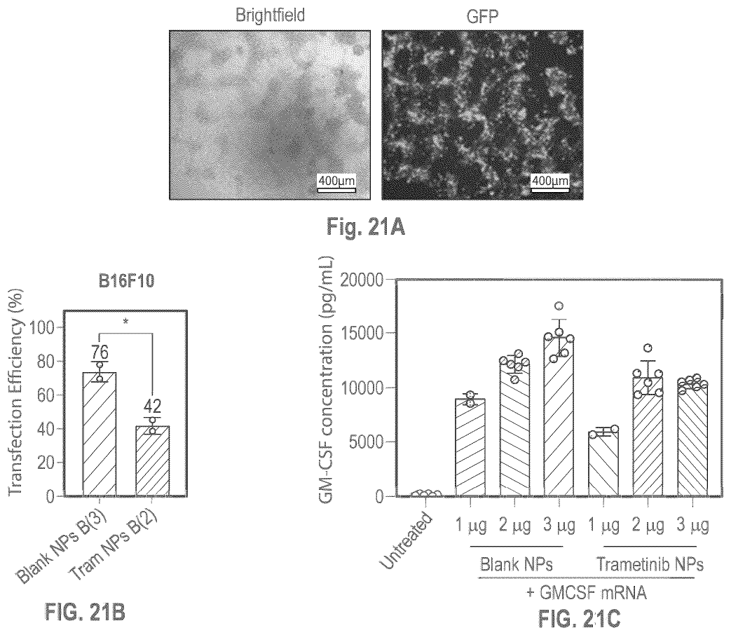

HEK293T transfection was highest at NP to mRNA Ratio A and a 1 μg total dose of mRNA. Observing cells under the microscope 48 hours post-transfection with GFP mRNA-NPs showed that the majority of cells were transfected and the transfection magnitude in these cells was relatively high (FIG. 21A).

In B16F10, it was observed that a higher NP to mRNA ratio and total dose of NPs was needed to achieve similarly high transfection efficiencies to HEK293T (FIG. 21B). 2 μg of mRNA for Tram-NPs was the best performing dose for transfecting B16F10 (FIG. 21B). Unloaded and Tram-NPs were then examined for their ability to transfect B16F10 with GM-CSF mRNA. Both NPs were able to efficiently transfect these cells to produce GM-CSF protein, with the maximum protein concentration for cells treated with the unloaded NPs and Tram-NPs being 146 and 109 times higher than the control, respectively (FIG. 21C). The ability for transfection by these NPs to result in such high levels of GM-CSF production is promising for their ability to produce these proteins in vivo to an amount that may trigger stronger immune responses at the tumor site.

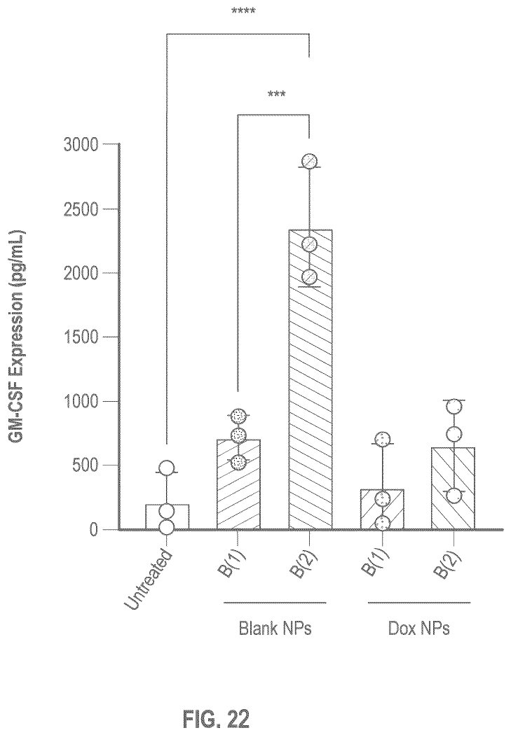

FIG. 22 shows that NPs loaded with GM-CSF mRNA or GM-CSF mRNA and doxorubicin resulted in increased GM-CSF expression compared to untreated cells.

### Example 8. NPs Inhibit Melanoma Tumor Growth and Prolong Survival in Mice

This Example demonstrates that NPs provide a therapeutic benefit in an in vivo murine melanoma model. Without wishing to be bound by theory, trametinib is expected to cause immunogenic cell death to cancer cells, and the secretion of GM-CSF by the transfected cells can facilitate the recruitment of immune cells and subsequently improve the overall immune response and enhance tumor eradication. In this study, transfection of tumors in mice were performed in situ, minimizing the steps necessary before vaccination. NPs were prepared as described in Examples 1, 3, and 7.

### Murine Tumor Treatment

6-8-week-old female C57BL/6 black mice (strain 000664) were purchased from Jackson Laboratory and used for all animal experiments. Water and food were provided ad libitum.

Syngeneic B16F10 tumor challenges were performed as follows: All tumor challenges were performed with low passage number (<10) B16F10 cells. B16F10 cells were thawed about a week before the tumor challenge and passaged as normal. On the day of tumor challenge, cells were detached with trypsin, quenched in cell culture media, and centrifuged at 4° C. Cell pellets were dispersed in cold serum-free cell culture media and put on ice until ready for injection. Mice were anesthetized with an intraperitoneal injection of ketamine/xylazine cocktail prior to tumor challenge. 1×10⁵ B16F10 cells were implanted subcutaneously into the right flank of mice using a syringe with a 26 G needle, where the cell solution formed a bleb under the skin. Tumor growth was observed two days a week until palpable tumors formed after 10 days.

Once palpable, tumors were treated with intratumoral injections of saline (untreated), non-drug loaded NPs (B NPs), trametinib-loaded NPs (T NPs), or trametinib-loaded NPs complexed with GM-CSF mRNA (T NPs+mRNA). All NP treatments were prepared at Ratio B (FIG. 27) and a total dose of 5 μg of mRNA was given per injection or an equivalent amount of nanoparticles in the case of the non-mRNA-loaded NP treatments. Assuming an average mouse weight of 20 g, the NP doses were 7.5 mg/kg and mRNA doses were 0.2875 mg/kg. 10×PBS was added to each treatment solution (final concentration 1×PBS) to isotonically balance the treatments to prevent cell lysis following administration. Treatments were given on days 10, 12, 14, 17, 19, and 21. Tumor growth was measured using digital calipers (United Scientific, cat. VCD001) by recording the greatest length and width across the tumor area. Tumor volume was calculated using the following formula: ½×L×W2. Mice were euthanized when tumors reached 20 mm in any dimension or if tumors became ulcerated. Mice were weighed using a balance on days tumor volumes were recorded.

### Results

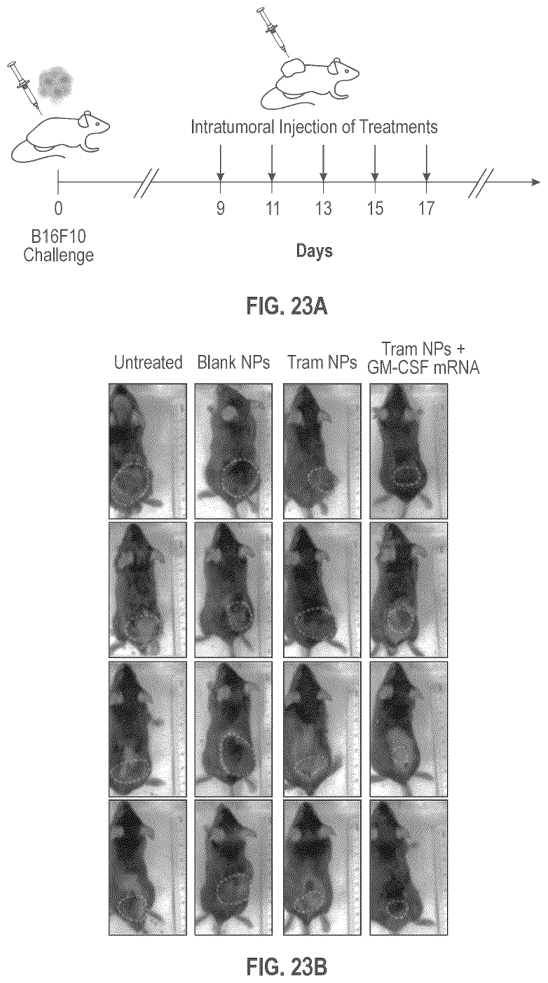

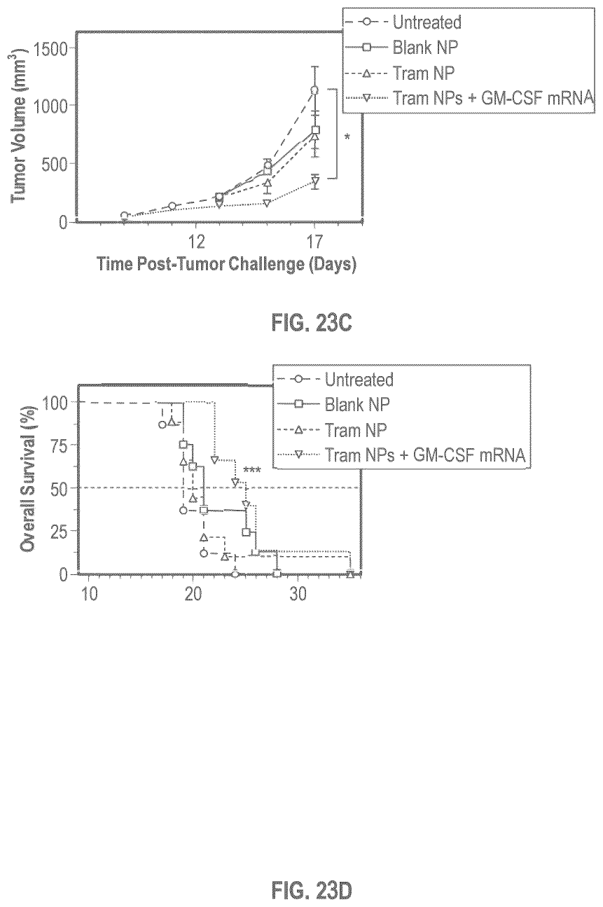

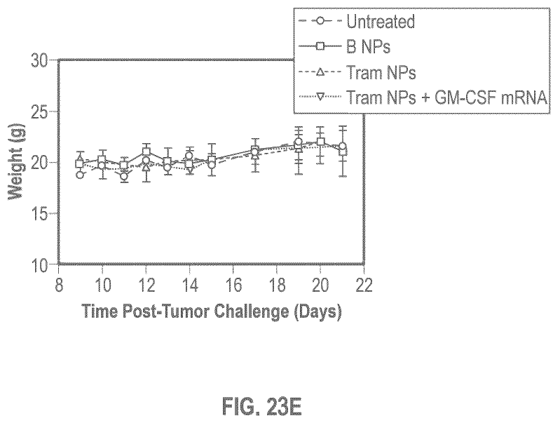

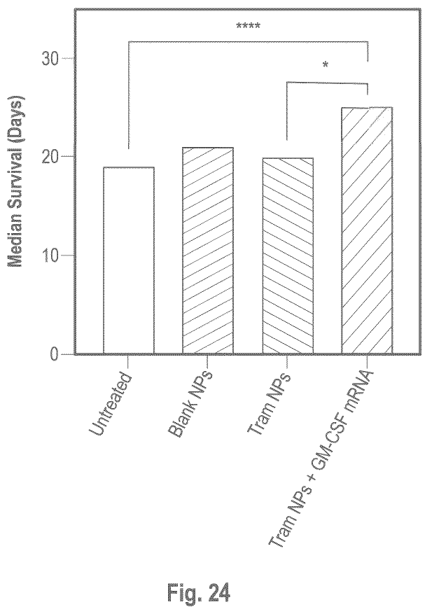

Mice were challenged with tumors and treated after 9 days of tumor growth when all tumors were palpable, with mice receiving intratumoral injections of either saline, unloaded NPs, Tram-NPs, and Tram-NPs complexed with GM-CSF mRNA every 2 days until endpoints were reached for each group (FIG. 23A). Representative photographs of tumor areas on day 17 are shown in FIG. 23B. NP treatment inhibited tumor growth over time, with the combination therapy resulting in significant delay of tumor growth (FIG. 23C). NP treatment improved survival of mice, with the combination therapy group demonstrating significant improvement over the untreated group (FIG. 23D). Median survival time of the Tram-NPs+GM-CSF mRNA group was improved by 32% (6 days) over the untreated group (FIG. 24). Throughout the duration of the experiment, mice weights were relatively stable, providing supporting evidence that NP therapy is relatively safe for in vivo intratumoral therapy (FIG. 23E). B16F10 is considered to be an immunologically “cold” tumor in regards to its low immune cell infiltration and tumor antigen expression, meaning that historically immunotherapy has struggled to bring about significant improvement in outcomes with this tumor model. The 32% survival improvement observed with this NP immunotherapy treatment is a promising initial result that may indicate this therapy could be effective in converting “cold” tumors to an overall more immunologically “hot” state for enhanced immune-mediated tumor clearance. Overall, the data from these preclinical experiments suggest the potential clinical relevance of this NP therapy for tumor treatment. These data show the ability of the NPs to co-deliver small molecules and genes, and their potential to work as a cancer vaccine in vivo.

### Example 9: Delivery of Plasmid DNA to Cells

This Example demonstrates that NPs can deliver plasmid DNA (pDNA) to cells in vitro. NPs were produced and loaded with pDNA as described in Example 1.

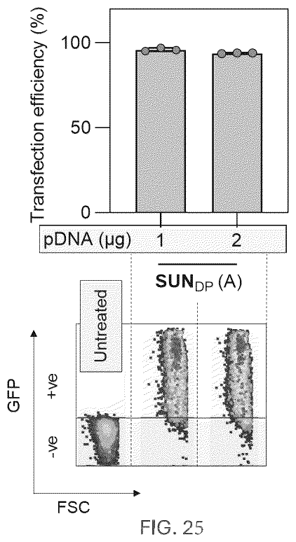

FIG. 25 show that NPs loaded with plasmid DNA encoding eGPF were successful in delivering pDNA to HEK293 cells and achieved high transfection efficiency, as demonstrated by flow cytometry.

### Example 10: Delivery of Luciferase mRNA to Cells

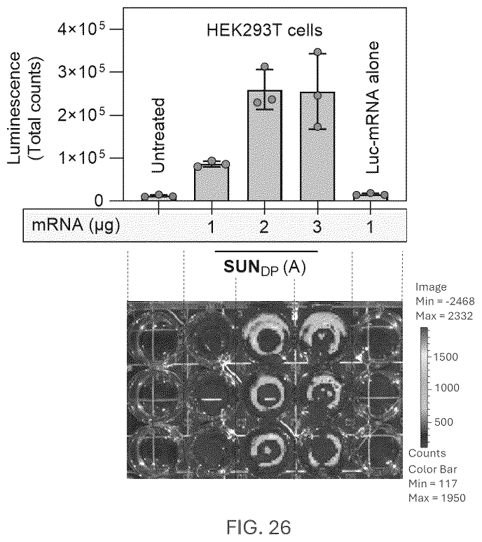

This Example demonstrates that NPs can deliver a high molecular weight mRNA, encoding luciferase, to cells in vitro. Nanoparticles were prepared as described in Example 1 and loaded with a luciferase encoding mRNA of 1921 nucleotides. NP solutions with 1 ug, 2 ug, or 3 ug were administered to the cells. FIG. 26 shows that cells were successfully transfected with luciferase. Untreated cells and cells with luciferase mRNA added showed no luciferase activity.

### Example 11: Characterization of Delivery of mRNA to Cells Using NPs

This Example describes further characterization of delivery of mRNA to cells using NPs, including delivery after various lengths of incubation, with different ratios of mRNA to NP, and with different total amounts of mRNA. NPs were prepared as described in Example 1, using Cy5 mRNA.

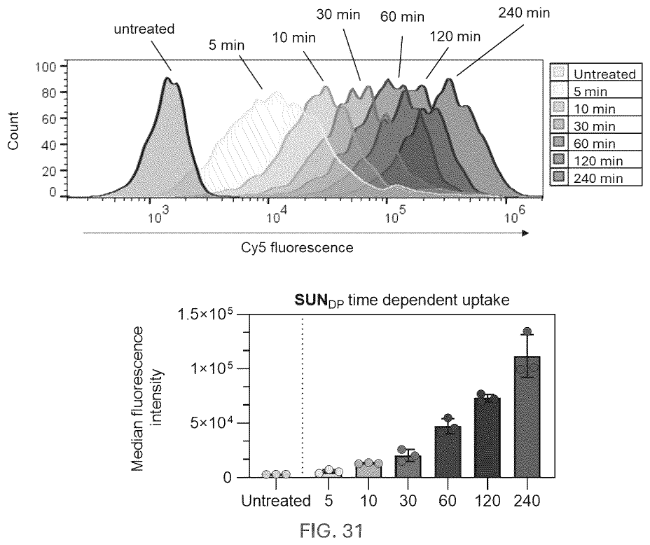

NPs were complexed to 1 μg of Cy5-Luc-mRNA at Ratio A (FIG. 27). HEK293T cells were incubated with the Cy5 mRNA NPs for 5, 10, 30, 60, 120, or 240 minutes. Expression of Cy5 was evaluated using flow cytometry. Cells incubated with Cy5 mRNA NPs for any length of time did express Cy5 (FIG. 31). Increasing the length of incubation increased the median Cy5 fluorescence intensity (FIG. 31).

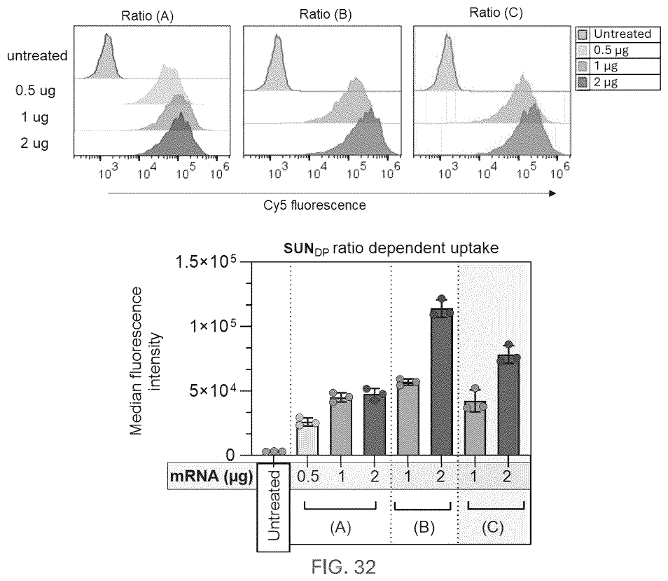

NPs were complexed to Cy5 mRNA at Ratio A, Ratio B, or Ratio C (FIG. 27), with a total amount of mRNA of 0.5, 1, or 2 ug (Ratio A) or 1 or 2 ug (Ratios B or C). HEK293T cells were incubated with the Cy5 mRNA NPs for 1 hour. Cells treated with the Cy5 mRNA NPs at any amount or ratio of mRNA did express Cy5 (FIG. 32). Treatment with higher amounts of mRNA generally resulted in higher median fluorescence intensity (FIG. 32).

This example demonstrates that NPs are able to successfully transfect cells with mRNA, and that parameters such as length of exposure and amount of mRNA per NP or total amount of mRNA can be titrated to affect the amount of transfection.

### Example 12: NPs Successfully Transfect Mouse and Human Cells

This Example describes delivery of mRNA to both human and mouse cell lines using NPs. eGFP mRNA NPs were produced as described in Example 1, at either Ratio A or Ratio B (FIG. 27) and with 1, 2, or 3 ug total mRNA (Ratio A) or 1.5 or 3 ug total mRNA (Ratio B).

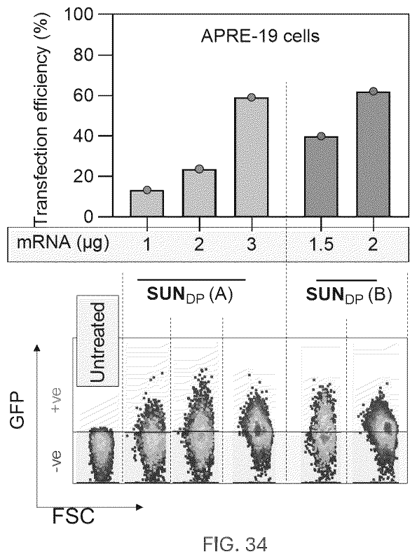

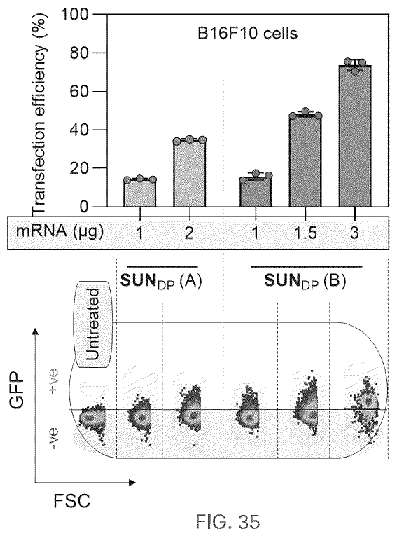

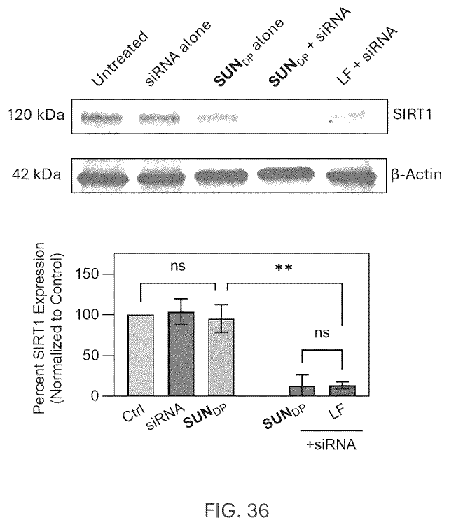

APRE-19 cells, which are human retinal pigment epithelial (RPE) cells, and B16F10 cells, which are murine melanoma cells, were treated with the eGFP mRNA NPs. NPs were able to transfect both APRE-19 cells (FIG. 34) and B16F10 cells (FIG. 35).

### Example 13: NPs Successfully Deliver siRNA

This Example describes the delivery of siRNA against SIRT1 to cells, which successfully lowered SIRT1 translation. NPs were prepared as described in Example 1 and complexed with siRNA. Cells were untreated, treated with SIRT1 siRNA alone, treated with NP (SUN_{DP}; formulation 66A in Table 2) alone, treated with NP and siRNA (SUN_{DP}+siRNA), or treated with LF (lipofection)+siRNA. The SIRT1 siRNA had the sequence of GCUGUACGAGGAGAUAUUUTT (SEQ ID NO: 2), in the 5′ to 3′ direction.

Treatment with NP+siRNA reduced SIRT1 protein in cells as much as delivery of the siRNA by lipofection. This demonstrates that the NPs can deliver siRNA as well as mRNA and pDNA.

### Example 14: Biodistribution of NPs

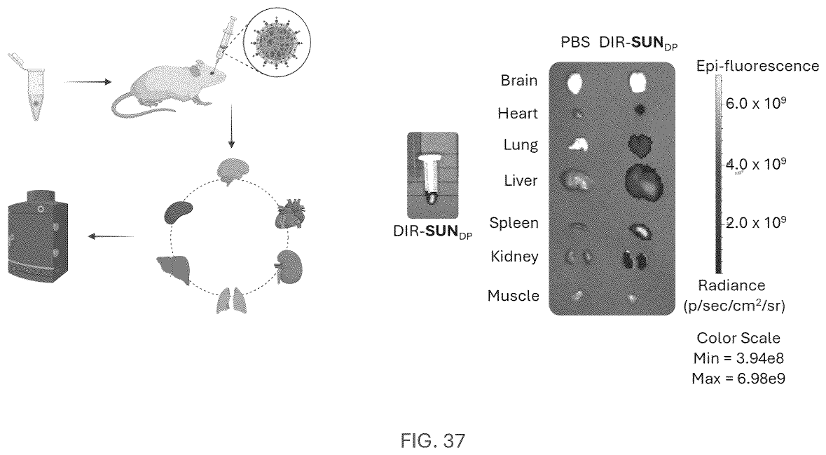

This Example describes the biodistribution of NPs containing DIR (1,1′-dioctadecyl-3,3,3′,3′-tetramethylindotricarbocyanine iodide), a lipophilic, near-infrared fluorescent cyanine dye. NPs loaded with DIR were administered to mice retroorbitally. The brain, heart, lung, liver, spleen, kidney, and muscle were collected 24 hours after injection. Imaging of these organs showed that the NPs containing DIR were present in the heart, lung, liver, spleen, and kidney (FIG. 37). Of these, strong localization was observed in the lung and liver and highest localization was in the spleen. This Example shows that NPs administered systemically are delivered to many of the major organs.

### OTHER EMBODIMENTS

Specific compositions comprising polymer-lipid-hybrid nanoparticles and methods of using same have been described. The scope of the invention should be defined by the claims. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the Applicant's information and belief. The Applicant makes no admission about the correctness of the contents or dates of these documents. Should there be a discrepancy between a date provided in this specification and the actual publication date, then the actual publication date shall control. Should there be a discrepancy between the scientific or technical teaching of a previous patent or publication and this specification, then the teaching of this specification and these claims shall control.

The foregoing written specification is considered sufficient to enable one skilled in the biomedical art to practice the present aspects and embodiments. The present aspects and embodiments are not to be limited in scope by examples provided, since the examples are intended as a single illustration of one aspect and other functionally equivalent embodiments are within the scope of the disclosure. Various modifications besides those shown and described herein will become apparent to those skilled in the biomedical art from the foregoing description and fall within the scope of the appended claims. The advantages and objects described herein are not necessarily encompassed by each embodiment. Those skilled in the biomedical art will recognize or be able to ascertain using no more than routine experimentation, many equivalents to the specific embodiments described herein. Such equivalents are intended to be encompassed by these claims.

1. A composition comprising:
(i) poly(lactic-co-glycolic acid) (PLGA);
(ii) polyethyleneimine (PEI); and
(iii) D-Lin-MC3-DMA (MC3).

2. (canceled)

3. A nanoparticle comprising:
(i) poly(lactic-co-glycolic acid) (PLGA) (ii) polyethyleneimine (PEI); and
(iii) D-Lin-MC3-DMA (MC3).

4. The nanoparticle of claim 3, which further comprises a nucleic acid.

5. The nanoparticle of claim 4, wherein:
(i) the nucleic acid is DNA or RNA;
(ii) the nucleic acid is mRNA and the mRNA encodes GM-CSF or a CRISPR-Cas protein;
(iii) the nucleic acid is located on the surface of the nanoparticle;
(iv) the nucleic acid complexes rapidly with the nanoparticle;
(v) the nanoparticle is capable of delivering the nucleic acid to a cell;
(vi) the nucleic acid is protected from nuclease digestion;
(vii) the nucleic acid is mRNA and the mRNA is complexed with the nanoparticle at a ratio of 1-10, 1-2, 2-4, or 4-10 molecules of mRNA per nanoparticle, or about 8 molecules of mRNA per nanoparticle, about 4 molecules of mRNA per nanoparticle, or about 2 molecules of mRNA per nanoparticle; and/or
(viii) the ratio of nanoparticle to nucleic acid by weight is 15+/−20%:1+/−20%, 30+/−20%:1+/−20%, or 60+/−20%:1+/−20%.

6.-8. (canceled)

9. The nanoparticle of claim 3, which further comprises a small molecule.

10. The nanoparticle of claim 9, wherein the small molecule is:
(i) encapsulated in the nanoparticle;
(ii) is a kinase inhibitor or intercalates with nucleic acid molecules.

11.-12. (canceled)

13. The nanoparticle of claim 3, wherein:
(i) the nanoparticle is in an aqueous solution or is a powder;
(ii) the nanoparticle has a mass ratio of about 15+/−20% PLGA:about 1.5+/−20% PEI:about 2+/−20% MC3;
(iii) the PLGA is poly(D,L-lactide-co-glycolide);
(iv) the PLGA has a ratio of lactic acid to glycolic acid monomers of 75:25, 50:50, or 85:15;
(v) the PLGA has a molecular weight of 38-54 kDa;
(vi) the PEI has an average molecular weight of 25 kDa; and/or
(vii) the PEI is linear or branched.

14.-21. (canceled)

22. The nanoparticle of claim 3, wherein:
(i) the nanoparticle has a diameter of 100-200 nm, 120-170 nm, 130-160 nm, 140-150 nm, or 144-148 nm, or about 146 nm;
(ii) the nanoparticle has a net positive charge;
(iii) the nanoparticle has a charge of 40-70 mV, 45-65 mV, 50-60 mV, or 50-55 mV, or about 53 mV;
(iv) the nanoparticle has a shelf life of at least 2 years;
(v) the nanoparticle maintains transfection efficiency when stored at −20° C. for at least 6 months, 12 months, 18 months, or 24 months;
(vi) the nanoparticle is double layered;
(vii) the nanoparticle does not comprise viral proteins, or fragments thereof; and/or
(viii) the nanoparticle is immunogenic, or the nanoparticle is non-immunogenic or does not induce an immune response.

23.-33. (canceled)

34. The nanoparticle of claim 3, wherein the nanoparticle;
(i) does not comprise cholesterol; and/or
(ii) comprises a small molecule and a nucleic acid.

35.-39. (canceled)

40. A kit comprising the nanoparticle of claim 3.

41. A kit comprising:
(i) poly(lactic-co-glycolic acid) (PLGA)
(ii) polyethyleneimine (PEI); and
(iii) D-Lin-MC3-DMA (MC3).

42. A container comprising the nanoparticle of claim 3.

43. A container comprising the nanoparticle of claim 3, and a nucleic acid molecule, wherein the composition or nanoparticle and nucleic acid molecule are a powder.

44. A method of storing the nanoparticle of claim 3, wherein the nanoparticle is a powder, comprising maintaining the container at a temperature of −20° C. for 2 years, wherein transfection activity does not drop more than 10%, 20%, 30%, 40%, or 50%.

45. A container or delivery device comprising the nanoparticle of claim 3, wherein the nanoparticle is a powder, and an aqueous solution comprising a nucleic acid molecule, wherein the composition or the nanoparticle are separated from the aqueous solution by a breakable septum.

46. (canceled)

47. A method of reconstituting the nanoparticle of claim 3, wherein the nanoparticle is a powder, the method comprising adding an aqueous solution to the powder.

48. A method of delivering a nucleic acid to a cell or tissue, the method comprising contacting the cell or tissue with the nanoparticle of claim 3, thereby delivering the nucleic acid.

49. A method of delivering a nucleic acid and a small molecule to a cell or tissue, the method comprising contacting the cell or tissue with the nanoparticle of claim 3, thereby delivering the nucleic acid.

50.-53. (canceled)

54. A method of delivering a small molecule to a cell or tissue, the method comprising contacting the cell or tissue with the nanoparticle of claim 3, thereby delivering the small molecule.

55.-59. (canceled)

60. A method of treating a disease or disorder, comprising administering the nanoparticle of claim 3 to a subject.

61.-62. (canceled)
